(19) United States
(12) Patent Application Publication
(10) Pub. No.: US 2010/0093725 A1
(43) Pub. Date: Apr. 15, 2010

(54) SEMI-SOLID FORMULATIONS OF PHOSPHOLIPASE ENZYME INHIBITORS

(75) Inventors: Mannching Sherry Ku, Thiells, NY (US); Frances Anne Donahue, Garfield, NJ (US); Weiyi Li, Pompton Plains, NJ (US)

Correspondence Address: WYETH LLC;PATENT LAW GROUP, 5 GIRALDA FARMS, MADISON, NJ, 07940, US

(73) Assignee: Wyeth

(21) Appl. No.: 12/513,101

(22) PCT Filed: Oct. 30, 2007
(86) PCT No.: PCT/US07/82985

§ 371 Date: Dec. 11, 2009

Related U.S. Application Data
(60) Provisional application No. 60/855,571, filed on Oct. 31, 2006.

(51) Int. Cl. A61K 31/404; A61K 31/5377; A61K 31/496; A61K 31/4709; A61P 29/00

(52) U.S. Cl. 514/235.2; 514/415; 514/254.09; 514/314

(57) ABSTRACT

The present invention is directed to semi-solid formulations of inhibitors of phospholipase enzymes, such as cytosolic PLA₂, compositions containing the same and processes for manufacture thereof.

### CROSS REFERENCE TO RELATED APPLICATIONS

This application claims the benefit of U.S. Provisional Application No. 60/855,571, filed on Oct. 31, 2006, which is incorporated herein by reference in its entirety.

### FIELD OF THE INVENTION

The present invention is directed to semi-solid formulations of inhibitors of phospholipase enzymes, such as cytosolic PLA₂, compositions containing the same and processes for manufacture thereof.

### BACKGROUND OF THE INVENTION

Leukotrienes and prostaglandins are important mediators of inflammation, each of which contributes to the development of an inflammatory response in a different way. Leukotrienes recruit inflammatory cells such as neutrophils to an inflamed site, promote the extravasation of these cells and stimulate release of superoxide and proteases, which damage the tissue. Leukotrienes also play a pathophysiological role in the hypersensitivity experienced by asthmatics {See, e.g. B. Samuelson et al., Science, 237:1171-76 (1987)). Prostaglandins enhance inflammation by increasing blood flow and therefore infiltration of leukocytes to inflamed sites. Prostaglandins also potentiate the pain response induced by stimuli.

Prostaglandins and leukotrienes are unstable and are not stored in cells, but are instead synthesized [W. L. Smith, Biochem. J., 259:315-324 (1989)] from arachidonic acid in response to stimuli. Prostaglandins are produced from arachidonic acid by the action of COX-1 and COX-2 enzymes. Arachidonic acid is also the substrate for the distinct enzyme pathway leading to the production of leukotrienes.

Arachidonic acid, which is fed into these two distinct inflammatory pathways, is released from the sn-2 position of membrane phospholipids by phospholipase A₂ enzymes (hereinafter PLA₂). The reaction catalyzed by PLA₂ is believed to represent the rate-limiting step in the process of lipid mediated biosynthesis and the production of inflammatory prostaglandins and leukotrienes. When the phospholipid substrate of PLA₂ is of the phosphotidyl choline class with an ether linkage in the sn-1 position, the lysophospholipid produced is the immediate precursor of platelet activating factor (hereafter called PAF), another potent mediator of inflammation [S. I. Wasserman, Hospital Practice, 15:49-58 (1988)].

Most anti-inflammatory therapies have focused on preventing production of either prostaglandins or leukotrienes from these distinct pathways, but not on all of them. For example, ibuprofen, aspirin, and indomethacin are all NSAIDs, which inhibit the production of prostaglandins by COX-1/COX-2 inhibition, but have no effect on the inflammatory production of leukotrienes from arachidonic acid in the other pathways. Conversely, zileuton inhibits only the pathway of conversion of arachidonic acid to leukotrienes, without affecting the production of prostaglandins. None of these widely-used anti-inflammatory agents affects the production of PAF.

Consequently the direct inhibition of the activity of PLA₂ has been suggested as a useful mechanism for a therapeutic agent, i.e., to interfere with the inflammatory response. [See, e.g., J. Chang et al, Biochem. Pharmacol., 36:2429-2436 (1987)].

A family of PLA₂ enzymes characterized by the presence of a secretion signal sequenced and ultimately secreted from the cell have been sequenced and structurally defined. These secreted PLA₂s have an approximately 14 kD molecular weight and contain seven disulfide bonds, which are necessary for activity. These PLA₂s are found in large quantities in mammalian pancreas, bee venom, and various snake venoms. [See, e.g., references 13-15 in Chang et al, cited above; and E. A. Dennis, Drug Devel. Res., 10:205-220 (1987).] However, the pancreatic enzyme is believed to serve a digestive function and, as such, should not be important in the production of the inflammatory mediators whose production must be tightly regulated.

The primary structure of the first human non-pancreatic PLA₂ has been determined. This non-pancreatic PLA₂ is found in platelets, synovial fluid, and spleen and is also a secreted enzyme. This enzyme is a member of the aforementioned family. [See J. J. Seilhamer et al., J. Biol. Chem., 264:5335-5338 (1989); R. M. Kramer et al., J. Biol. Chem., 264:5768-5775 (1989); and A. Kando et al., Biochem. Biophys. Res. Comm., 163:42-48 (1989)]. However, it is doubtful that this enzyme is important in the synthesis of prostaglandins, leukotrienes and PAF, since the non-pancreatic PLA₂ is an extracellular protein, which would be difficult to regulate, and the next enzymes in the biosynthetic pathways for these compounds are intracellular proteins. Moreover, there is evidence that PLA₂ is regulated by protein kinase C and G proteins [R. Burch and J. Axelrod, Proc. Natl. Acad. Sci. U.S.A., 84:6374-6378 (1989)], which are cytosolic proteins, which must act on intracellular proteins. It would be impossible for the non-pancreatic PLA₂ to function in the cytosol, since the high reduction potential would reduce the disulfide bonds and inactivate the enzyme.

A murine PLA₂ has been identified in the murine macrophage cell line, designated RAW 264.7. A specific activity of 2 mols/min/mg, resistant to reducing conditions, was reported to be associated with the approximately 60 kD molecule. However, this protein was not purified to homogeneity. [See, C. C. Leslie et al., Biochem. Biophys. Acta., 963:476-492 (1988)]. The references cited above are incorporated by reference herein for information pertaining to the function of the phospholipase enzymes, particularly PLA₂.

A cytosolic phospholipase A₂ alpha (hereinafter “cPLA₂α”) has also been identified and cloned. See, U.S. Pat. Nos. 5,322,776 and 5,354,677, which are incorporated herein in their entirety. The enzyme of these patents is an intracellular PLA₂ enzyme, purified from its natural source or otherwise produced in purified form, which functions intracellularly to produce arachidonic acid in response to inflammatory stimuli.

In addition to the identification of several phospholipase enzymes, efforts have been spent in identifying chemical inhibitors of the action of specific phospholipase enzymes, which inhibitors could be used to treat inflammatory conditions, particularly where inhibition of production of prostaglandins, leukotrienes and PAF are all desired results. Such inhibitors are disclosed, for example, in U.S. Pat. No. 6,797,708 and U.S. patent application Ser. No. 11/442,199 (filed May 26, 2006), each of which is incorporated herein by reference in their entireties.

Given the importance of these compounds as pharmaceutical agents, it can be seen that effective formulations for delivery of the compounds, including those having improved bioavailability, are of great import, and there is an ongoing need for such new formulations.

### SUMMARY OF THE INVENTION

The invention provides pharmaceutical compositions comprising:

a) a pharmaceutically effective amount of an active pharmacological agent having Formula I:

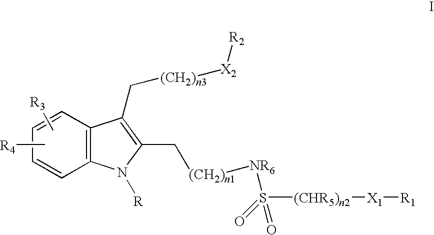

or a pharmaceutically acceptable salt thereof, wherein R, R₁, R₂, R₃, R₄, R₆, X₁, X₂, n₁, n₂, and n₃ are defined as described herein; and

b) a carrier or excipient system comprising a viscosity builder, a solubilizer, a diluent, and a stabilizer.

In some embodiments, the present invention provides pharmaceutical compositions comprising:

a) a pharmaceutically effective amount of an active pharmacological agent having the Formula II:

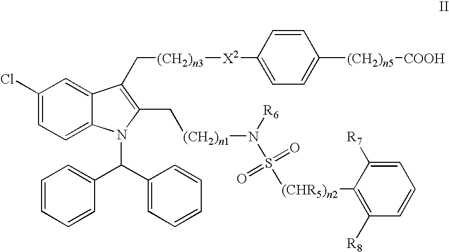

and pharmaceutically acceptable salts thereof, wherein R₅, R₆, R₇, R₈, X², nᵢ, n₂, n₃, and n₅ are defined as described herein; and

b) a carrier or excipient system comprising a viscosity builder, a solubilizer, a diluent, and a stabilizer.

The invention further provides processes for preparing the pharmaceutical compositions and dosage forms of the invention, and products of the processes.

### BRIEF DESCRIPTION OF THE DRAWINGS

FIG. 1 is a graph depicting the dissolution profile of a formulation according to the invention at different pH.

FIG. 2 is a graph depicting the dissolution profile in simulated fed and fasted state media of a formulation according to the invention.

### DETAILED DESCRIPTION OF THE INVENTION

In one aspect, the invention provides pharmaceutical composition comprising:
a) a pharmaceutically effective amount of an active pharmacological agent having Formula I:

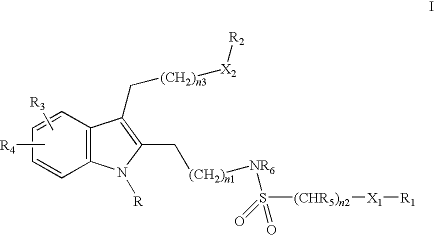

or a pharmaceutically acceptable salt thereof, wherein:
R is selected from the formulae —(CH₂)ₙ-A, —(CH₂)ₙ—S-A, and —(CH₂)ₙ—O-A, wherein A is selected from the moieties:

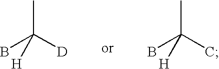

wherein
D is C₁-C₆ alkyl, C₁-C₆ alkoxy, C₃-C₆ cylcoalkyl, —CF₃, or —(CH₂)₁₋₃—CF₃;

B and C are independently selected from phenyl, pyridinyl, pyrimidinyl, furyl, thienyl and pyrrolyl groups, each optionally substituted by from 1 to 3, preferably 1 to 2, substituents selected independently from halogen, —CN, —CHO, —CF₃, —OCF₃, —OH, C₁-C₆ alkyl, C₁-C₆ alkoxy, —NH₂, —N(C₁-C₆ alkyl)₂, —NH(C₁-C₆ alkyl), —NH—C(O)—(C₁-C₆ alkyl), —NO₂, or by a 5- or 6-membered heterocyclic or heteroaromatic ring containing 1 or 2 heteroatoms selected from O, N, and S; or
n is an integer from 0 to 3;
n₁ is an integer from 1 to 3;
n₂ is an integer from 0 to 4;
n₃ is an integer from 0 to 3;
n₄ is an integer from 0 to 2;
X₁ is selected from a chemical bond, —S—, —O—, —S(O)—, —S(O)₂—, —NH—, —C═C—,

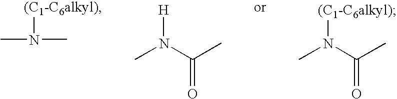

R₁ is selected from C₁-C₆ alkyl, C₁-C₆ fluorinated alkyl, C₃-C₆ cycloalkyl, tetrahydropyranyl, camphoryl, adamantyl, —CN, —N(C₁-C₆ alkyl)₂, phenyl, pyridinyl, pyrimidinyl, furyl, thienyl, napthyl, morpholinyl, triazolyl, pyrazolyl, piperidinyl, pyrrolidinyl, imidazolyl, piperizinyl, thiazolidinyl, thiomorpholinyl, tetrazolyl, indolyl, benzoxazolyl, benzofuranyl, imidazolidine-2-thionyl, 7,7-dimethyl-bicyclo[2.2.1]heptan-2-onyl, benzo[1,2,5]oxadiazolyl, 2-oxa-5-aza-bicyclo[2.2.1]heptanyl, piperazin-2-onyl and pyrrolyl groups, each optionally substituted by from 1 to 3, preferably 1 to 2, substituents independently selected from halogen, —CN, —CHO, —CF₃, —OCF₃, —OH, C₁-C₆ alkyl, C₁-C₆ alkoxy, —NH₂, —N(C₁-C₆alkyl)₂, —NH(C₁-C₆ alkyl), —NH—C(O)—(C₁-C₆ alkyl), —NO₂, —SO₂(C₁-C₃ alkyl), —SO₂NH₂, —SO₂NH(C₁-C₃ alkyl), —SO₂N(C₁-C₃ alkyl)₂, —COOH, —CH₂—COOH, —CH₂—N(C₁-C₆ alkyl), —CH₂—N(C₁-C₆ alkyl)₂, —CH₂—NH₂, pyridinyl, 2-methyl-thiazolyl, morpholino, 1-chloro-2-methyl-propyl, C₁-C₆ thioalkyl, phenyl (further optionally substituted with one or more (e.g., 1-5, 1-4, 1-3, or 1-2) halogens), dialkylamino, —CN or —OCF₃), benzyloxy, —(C₁-C₃ alkyl)C(O)CH₃, —(C₁-C₃ alkyl)OCH₃, —C(O)NH₂, or

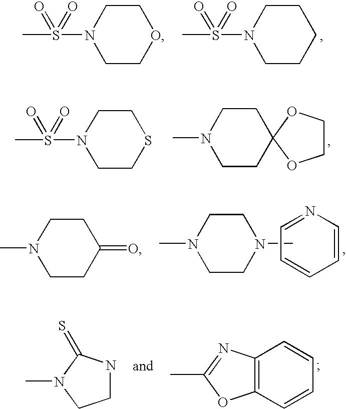

X₂ is selected from —O—, —CH₂—, —S—, —SO—, —SO₂—, —NH—, —C(O)—,

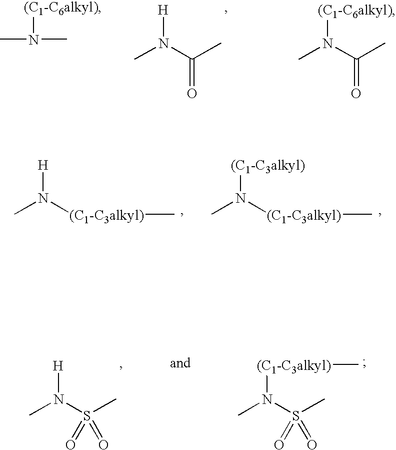

R₂ is a ring moiety selected from phenyl, pyridinyl, pyrimidinyl, furyl, thienyl and pyrrolyl groups, the ring moiety being substituted by a group of the formula —(CH₂)ₙ₄—CO₂H or a pharmaceutically acceptable acid mimic or mimetic; and also optionally substituted by 1 or 2 additional substituents independently selected from halogen, —CN, —CHO, —CF₃, —OCF₃, —OH, C₁-C₆ alkyl, C₁-C₆ alkoxy, C₁-C₆ thioalkyl, —NH₂, —N(C₁-C₆ alkyl)₂, —NH(C₁-C₆ alkyl), —NH—C(O)—(C₁-C₆ alkyl), and —NO₂;

R₃ is selected from H, halogen, —CN, —CHO, —CF₃, —OCF₃, —OH, C₁-C₆ alkyl, C₁-C₆ alkoxy, C₁-C₆ thioalkyl, —NH₂, —N(C₁-C₆ alkyl)₂, —NH(C₁-C₆ alkyl), —NH—C(O)—(C₁-C₆ alkyl), and —NO₂;

R₄ is selected from H, halogen, —CN, —CHO, —CF₃, —OCF₃, —OH, C₁-C₆ alkyl, C₁-C₆ alkoxy, C₁-C₆ thioalkyl, —NH₂, —N(C₁-C₆ alkyl)₂, —NH(C₁-C₆ alkyl), —NH—C(O)—(C₁-C₆ alkyl), —NO₂, —NH—C(O)—N(C₁-C₃ alkyl)₂, —NH—C(O)—NH(C₁-C₃ alkyl), —NH—C(O)—O—(C₁-C₃ alkyl), —SO₂—C₁-C₆ alkyl, —S—C₃-C₆ cycloalkyl, —S—CH₂—C₃-C₆ cycloalkyl, —SO₂—C₃-C₆ cycloalkyl, —SO₂—CH₂—C₃-C₆ cycloalkyl, C₃-C₆ cycloalkyl, —CH₂—C₃-C₆ cycloalkyl, —O—C₃-C₆ cycloalkyl, —O—CH₂—C₃-C₆ cycloalkyl, phenyl, benzyl, benzyloxy, morpholino, pyrrolidino, piperidinyl, piperizinyl, furanyl, thienyl, imidazolyl, tetrazolyl, pyrazinyl, pyrazolonyl, pyrazolyl, oxazolyl, and isoxazolyl, the rings of each of these R₄ groups each being optionally substituted by from 1 to 3 substituents selected from the group of halogen, —CN, —CHO, —CF₃, —OH, C₁-C₆ alkyl, C₁-C₆ alkoxy, —NH₂, —N(C₁-C₆ alkyl)₂, —NH(C₁-C₆ alkyl), —NH—C(O)—(C₁-C₆ alkyl), —NO₂, —SO₂(C₁-C₃ alkyl), —SO₂NH(C₁-C₃ alkyl), —SO₂N(C₁-C₃ alkyl)₂, and —OCF₃;

each R5 is independently H or C1-3 alkyl; and

R₆ is H or C₁₋₆ alkyl; and

b) a carrier or excipient system comprising:
i) about 15 to about 25% a viscosity builder by weight of the composition;
ii) about 5 to about 15% a solubilizer by weight of the composition; and
iii) about 10 to about 50% a diluent by weight of the composition; and
iv) about 1 to about 10% a stabilizer by weight of the composition.

In some aspects, the invention provides the pharmaceutical composition wherein

R₁ is optionally substituted phenyl; and

R is

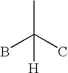

where B and C are phenyl.

In one aspect, this invention provides pharmaceutical compositions comprising:

a) a pharmaceutically effective amount of an active pharmacological agent having the Formula II:

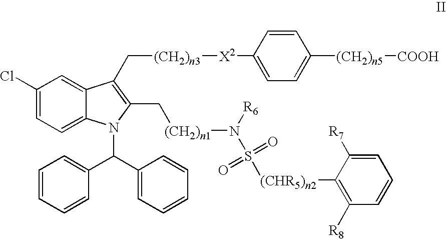

or a pharmaceutically acceptable salt thereof, wherein:

n₁ is 1 or 2;

n₂ is 1 or 2;

n₃ is 1 or 2;

n₅ is 0, 1 or 2;

X² is O, —CH₂— or SO₂;

each R₅ is independently H or C₁₋₃ alkyl;

R₆ is H or C₁₋₆ alkyl;

R₇ is selected from the group consisting of —OH, benzyloxy, —CH₃, —CF₃, —OCF₃, —C₁₋₃ alkoxy, halogen, —CHO, —CO(C₁₋₃ alkyl), —CO(OC₁₋₃ alkyl), quinoline-5-yl, 3,5-dimethylisoxazol-4-yl, thiophene-3-yl, pyridin-4-yl, pyridine-3-yl, —CH₂-Q, and phenyl optionally substituted by from one to three independently selected R₃₀ groups;

R₈ is selected from the group consisting of H, —OH, —NO₂, —CF₃, —OCF₃, C₁₋₃ alkoxy, halogen, —CO(C₁₋₃ alkyl), —CO(OC₁₋₃ alkyl), quinoline-5-yl, 3,5-dimethylisoxazol-4-yl, thiophene-3-yl, —CH₂-Q, and phenyl substituted by from one to three independently selected R₃₀ groups;

Q is OH, dialkylamino,

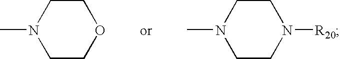

R₂₀ is selected from the group consisting of H, C₁₋₃ alkyl, and —CO(C₁₋₃ alkyl); and

R₃₀ is selected from the group consisting of dialkylamino, —CN and —OCF₃; provided that:

i) when each R₅ is H, R₆ is H, n₅ is 0, and R₈ is H, then R₇ cannot be chlorine;

ii) when each R₅ is H, R₆ is H, n₅ is 0, X² is O or —CH₂—, and R₈ is H, then R₇ cannot be CH₃;

iii) when each R₅ is H, and R₆ is H, then R₇ and R₈ cannot both be fluorine;

iv) when each R₅ is H, R₆ is H, and X² is O, then R₇ and R₈ cannot both be chlorine;

v) when each R₅ is H, R₆ is H, X² is O, and R₈ is NO₂, then R₇ cannot be fluorine; and

vi) when each R₅ is H, R₆ is H, X² is SO₂, and R₈ is H, then R₇ cannot be fluorine or chlorine; and

b) a carrier or excipient system comprising:
i) about 15 to about 25% a viscosity builder by weight of the composition;
ii) about 5 to about 15% a solubilizer by weight of the composition; and
iii) about 10 to about 50% a diluent by weight of the composition; and
iv) about 1 to about 10% a stabilizer by weight of the composition.

In some embodiments, the compound of Formula I or Formula II has the Formula III:

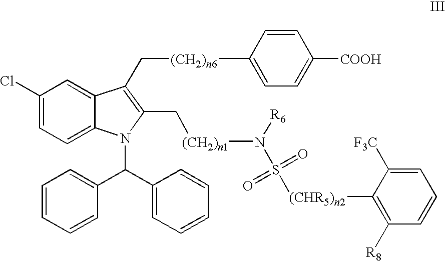

or a pharmaceutically acceptable salt thereof, wherein:

n₁ is 1 or 2;

n₂ is 1 or 2;

n₆ is 1 or 2;

R₅ is H or CH₃;

R₆ is H or C₁₋₆ alkyl; and

R₈ is selected from the group consisting of H, —OH, —NO₂, —CF₃, —OCF₃, —OCH₃, halogen, —COCH₃, —COOCH₃, dimethylamino, diethylamino, and —CN.

In some further embodiments, the compound of Formula I or Formula II is 4-(3-{5-chloro-1-(diphenylmethyl)-2-[2-({[2-(trifluoromethyl)benzyl]sulfonyl}amino)ethyl]-1H-indol-3-yl}propyl)benzoic acid or a pharmaceutically acceptable salt thereof.

It will be understood that the C₁-C₆ fluorinated alkyl groups in the definition of R₁ may be any alkyl group of 1 to 6 carbon atoms with any amount of fluorine substitution including, but not limited to, —CF₃, alkyl chains of 1 to 6 carbon atoms terminating in a trifluoromethyl group, —CF₂CF₃, etc.

As used herein, the terms “heterocyclic” or “heterocyclyl” refer to a saturated or partially unsaturated (nonaromatic) monocyclic, bicyclic, tricyclic or other polycyclic ring system having 1-4 ring heteroatoms if monocyclic, 1-8 ring heteroatoms if bicyclic, or 1-10 ring heteroatoms if tricyclic, each of said heteroatoms being independently selected from O, N, and S (and mono and dioxides thereof, e.g., N→O—, S(O), SO₂. A ring heteroatom or a ring carbon can serve as the point of attachment of the heterocyclic ring to another moiety. Any atom can be substituted, e.g., by one or more substituents. Heterocyclyl groups can include, e.g. and without limitation, tetrahydropyranyl, piperidyl (piperidine), piperazinyl, morpholinyl (morpholino), thiomorpholinyl, pyrrolinyl, and pyrrolidinyl.

The term “heteroaromatic” refers to an aromatic monocyclic, bicyclic, tricyclic, or other polycyclic hydrocarbon group having 1-4 ring heteroatoms if monocyclic, 1-8 ring heteroatoms if bicyclic, or 1-10 ring heteroatoms if tricyclic, each of said heteroatoms being independently selected from O, N, and S (and mono and dioxides thereof, e.g., N→O⁻, S(O), SO₂). Any atom can be substituted, e.g., by one or more substituents. Heteroaromatic rings can include, e.g. and without limitation, pyridinyl, thiophenyl (thienyl), furyl (furanyl), imidazolyl, indolyl, isoquinolyl, quinolyl and pyrrolyl.

Pharmaceutically acceptable acid mimics or mimetics useful in the compounds of this invention include those wherein R₂ is selected from the group of:

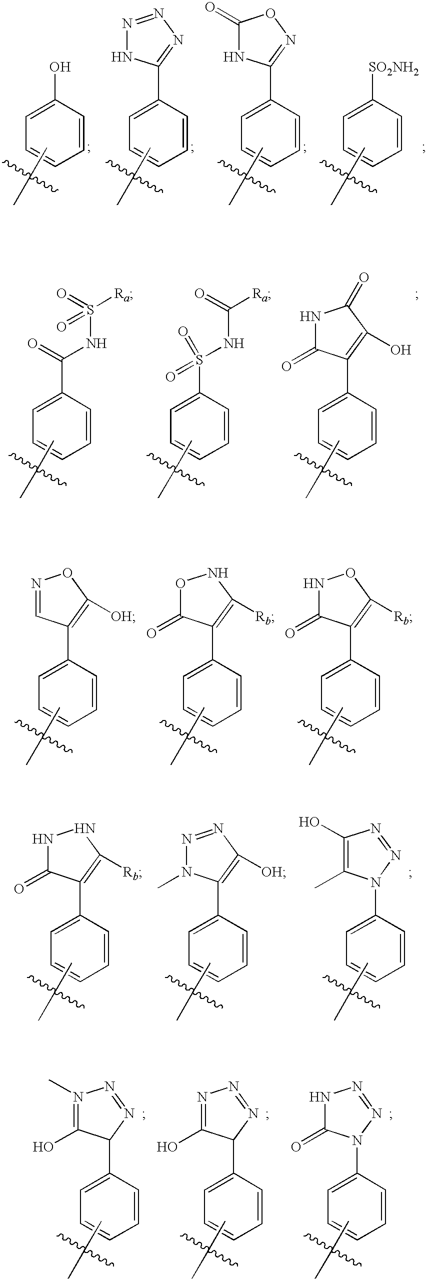

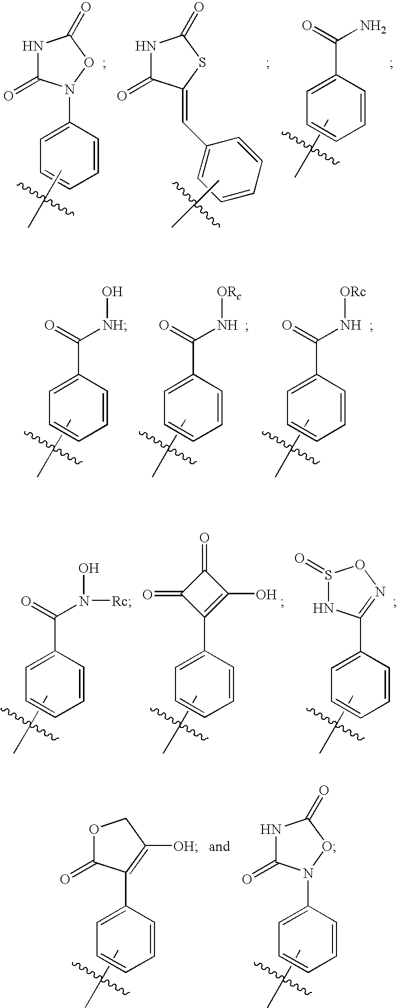

wherein Rₐ is selected from —CF₃, —CH₃, phenyl, and benzyl, with the phenyl or benzyl groups being optionally substituted by from 1 to 3 groups selected from C₁-C₆ alkyl, C₁-C₆ alkoxy, C₁-C₆ thioalkyl, —CF₃, halogen, —OH, and —COOH; R_{b} is selected from —CF₃, —CH₃, —NH₂, phenyl, and benzyl, with the phenyl or benzyl groups being optionally substituted by from 1 to 3 groups selected from C₁-C₆ alkyl, C₁-C₆ alkoxy, C₁-C₆ thioalkyl, —CF₃, halogen, —OH, and —COOH; and R_{c} is selected from —CF₃ and C₁-C₆ alkyl.

Those of skill in the art will be able to readily ascertain pharmaceutically effective amounts of said active pharmacological agent. Generally, the active pharmacological agent is present in the composition in an amount of from about 0.1% to about 25% by weight of the composition.

In some embodiments, the invention provides unit dosage forms containing the compositions of the invention. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Thus, the unit dosage forms formulations of the present invention include any conventionally used forms, including capsules, gels, oral liquids, and the like. In some embodiments, the unit dosage form is a capsule.

As will be recognized, a unit dosage form, such as a capsule, tablet, or other dosage form, will generally contain a pharmaceutically effective amount of the active pharmacological agent. As will be recognized, the pharmacological agent can be effective over a wide dosage range, and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

Generally, on a weight basis, the pharmaceutically effective amount is from about 1 mg to about 125 mg of active pharmacological agent. Thus, the unit dosage forms of the invention can contain various doses of the active pharmacological agent, for example approximate doses of 5, 10, 25, 50, 75, and 100 mg, as well as others. Accordingly, the invention includes dosage forms that contain pharmaceutical compositions of the invention, that include from about 3 mg to about 7 mg of active pharmacological agent, from about 8 mg to about 12 mg of active pharmacological agent, from about 13 mg to about 19 mg of active pharmacological agent, from about 20 mg to about 30 mg of active pharmacological agent, from about 31 mg to about 60 mg of active pharmacological agent, from about 61 mg to about 80 mg of active pharmacological agent, and from about 81 mg to about 110 mg of active pharmacological agent. One preferred embodiment is a 500 mg capsule containing 100 mg of pharmacologically active agent (i.e. 500 mg of a composition of the invention containing 20% pharmacologically active agent by weight of the pharmaceutical composition).

Generally, the compositions of the invention include one or more viscosity builders, i.e., compounds that increase the viscosity of the composition. Generally, the viscosity builder is present in an amount of from about 15% to about 25% by weight of the composition. Any suitable viscosity builder known in the art can be used. In some embodiments, the viscosity builder is selected from PEG 1000, PEG 1500, Gelucire 44/14, Gelucire 50/13, and mixtures thereof. In some embodiments, the viscosity builder comprises or consists of PEG 1000.

Generally, the compositions of the invention include one or more solubilizers. Generally, the solubilizer is present in an amount of from about 5% to about 15% by weight of the composition. Solubilizers include, for example, surfactants. Any suitable solubilizer known in the art can be used. In some embodiments, the solubilizer is selected from polysorbate 80, polyoxyl 40 hydrogenated castor oil, polyoxyl 35 castor oil, and mixtures thereof. In some embodiments, the solubilizer comprises or consists of polysorbate 80.

Generally, the compositions of the invention include a diluent. Generally, the diluent is present in an amount of from about 10% to about 50% by weight of the composition. Any suitable diluent and/or solvent, or combination thereof, may be used for the diluent. In some embodiments, the diluents are selected from PEG 400, propylene glycol, propylene carbonate, triacetin, and mixtures thereof. In some further embodiments, the diluent comprises or consists of PEG 400.

Generally, the compositions of the invention include one or more stabilizers. Generally, the stabilizer is present in an amount of from about 1% to about 10% by weight of the composition. Any suitable stabilizer known in the art can be used. Stabilizers include, for example, dispersing agents. In some embodiments, the stabilizer is selected from the polyvinylpyrrolidones (PVP) and mixtures thereof. In some embodiments, the PVP is selected from PVP-K-17, PVP-K-12, and mixtures thereof. In some further embodiments, the stabilizer is PVP-K-17.

In some embodiments of the invention, the pharmaceutical composition comprises the pharmacologically active agent and the carrier or excipient system wherein:

i) the viscosity builder is selected from the group consisting of PEG 1000, PEG 1500, Gelucire 44/14, Gelucire 50/13, and mixtures thereof;

ii) the solubilizer is selected from the group consisting of polysorbate 80, polyoxyl 40 hydrogenated castor oil, polyoxyl 35 castor oil, and mixtures thereof;

iii) the diluent is selected from the group consisting of PEG 400, propylene glycol, propylene carbonate, triacetin, and mixtures thereof; and

v) the stabilizer is a polyvinylpyrrolidone.

In some further embodiments, the pharmaceutical composition comprises the pharmacologically active agent and the carrier or excipient system, which comprises:

i) PEG 1000 in an amount of from about 15% to about 25% by weight of the composition;

ii) polysorbate 80 in an amount of from about 5% to about 15% by weight of the composition;

iii) PEG 400 in an amount of from about 10% to about 50% by weight of the composition; and

iv) PVP K-17 in an amount of from about 1% to about 10% by weight of the composition.

In one particular embodiment, the invention provides a pharmaceutical composition comprising:

a) about 20% by weight of the composition of the active pharmacological agent 4-(3-{5-chloro-1-(diphenylmethyl)-2-[2-({[2(trifluoromethyl)benzyl]sulfonyl}amino)ethyl]-1H-indol-3-yl}propyl)benzoic acid or a pharmaceutically acceptable salt thereof; and

b) a carrier or excipient system comprising:
i) PEG 1000 in an amount of about 20% by weight of the composition;
ii) polysorbate 80 in an amount of about 10% by weight of the composition;
iii) PEG 400 in an amount of about 40% by weight of the composition; and
iv) PVP K-17 in an amount of about 10% by weight of the composition.

In some embodiments, the invention provides unit dosage forms comprising a pharmaceutical composition as described above, wherein the composition contains about 100 mg of the active pharmacological agent. As discussed above, other doses can be made into unit dosage forms as is well known to those of skill in the art.

Because of the semi-solid nature of the resulting pharmaceutical composition, unit dosage forms such as capsules are well suited for administering the pharmaceutical composition to a patient. The invention also includes methods of preparing the pharmaceutical composition for administration, particularly via a capsule unit dosage form.

In some embodiments, the invention provides a process for preparing a pharmaceutical composition as described above, comprising the steps of:
(1) mixing a viscosity builder, a solubilizer and a diluent to produce a first homogenous solution;
(2) slowly adding a stabilizer until dissolved to form a second homogenous solution;
(3) slowly adding the pharmacologically active agent to said cooled second homogenous solution; and
(4) mixing with sufficient heating until the pharmacologically active agent is dissolved to produce a third homogenous solution.

To facilitate the mixing and dissolution, the viscosity builder, solubilizer, and diluent can be heated, for example to from about 90° C. to about 100° C., for example to about 95° C., while mixing. In some embodiments, the temperature is maintained at 95 +/−5° C.

When the resultant first homogenous solution is heated, the second homogenous solution can be cooled (e.g., to from about 80° C. to about 90° C. or to about 85° C.) prior to the addition of the pharmaceutically active agent. In some embodiments, the temperature is maintained at 85 +/−5° C.

As discussed above, the resultant product is suitable for administration via a capsule. Accordingly, the process for preparing the pharmaceutical composition may further include encapsulating at least a portion of the second homogenous solution into one or more unit dosage capsule forms. Those of skill in the art will appreciate that any suitable encapsulation technique may be used.

In some embodiments, the third homogenous solution is cooled, preferably to about 40° C., prior to encapsulation to enhance its handling and to prevent melting or dissolution of the encapsulating material.

Those of skill in the art will readily recognize that simple modification of the steps outlined above, and the relative amounts of each of the components, will result in formation of a final product of desired size, strength and composition. Accordingly, the process described above can be used to make any of the pharmaceutical compositions described herein.

In particular, the process is useful in making such pharmaceutical compositions where the pharmaceutically effective amount of the active pharmacological agent is about 0.1 to about 20% by weight of the composition.

The process is also useful in making such pharmaceutical compositions where the viscosity builder is selected from the group consisting of PEG 1000, PEG 1500, Gelucire 44/14, Gelucire 50/13, and mixtures thereof, for example, when the viscosity builder is PEG 1000.

The process is also useful in making such pharmaceutical compositions where the solubilizer is selected from the group consisting of polysorbate 80, polyoxyl 40 hydrogenated castor oil, polyoxyl 35 castor oil, and mixtures thereof, for example, where the solubilizer is polysorbate 80.

The process is also useful in making such pharmaceutical compositions where the diluent is selected from the group consisting of PEG 400, propylene glycol, propylene carbonate, triacetin, and mixtures thereof, for example, where the diluent PEG 400.

The process is also useful in making such pharmaceutical compositions where the stabilizer is a polyvinylpyrrolidone, for example, where the stabilizer is selected from polyvinylpyrrolidone 12 (PVP-K-12), polyvinylpyrrolidone 17 (PVP-K-17) and mixtures thereof.

The process is also useful in making such pharmaceutical compositions where the pharmaceutical composition comprises a pharmacologically active agent and a carrier or excipient system wherein:
i) the viscosity builder is selected from the group consisting of PEG 1000, PEG 1500, Gelucire 44/14, Gelucire 50/13, and mixtures thereof;
ii) the solubilizer is selected from the group consisting of polysorbate 80, polyoxyl 40 hydrogenated castor oil, polyoxyl 35 castor oil, and mixtures thereof;
iii) the diluent is selected from the group consisting of PEG 400, propylene glycol, propylene carbonate, triacetin, and mixtures thereof; and
iv) the stabilizer is a polyvinylpyrrolidone.

For example, the process is useful in making such pharmaceutical compositions where the pharmaceutical composition comprising a pharmacologically active agent and a carrier or excipient system comprising:
i) PEG 1000 in an amount of from about 15% to about 25% by weight of the composition;
ii) polysorbate 80 in an amount of from about 5% to about 15% by weight of the composition;
iii) PEG 400 in an amount of from about 10% to about 50% by weight of the composition; and
iv) PVP K-17 in an amount of from about 1% to about 10% by weight of the composition.

As described above, the process can be used to make various sized unit dosage forms. Generally, the dosage forms contain from about 1 mg to about 125 mg of active pharmacological agent. Typical unit dosage forms will contain about 5, 10, 25, 50, 75 or 100 mg active agent. Accordingly, the invention includes dosage forms comprising a pharmaceutical composition of the invention, wherein the composition comprises from about 3 mg to about 7 mg of active pharmacological agent, from about 8 mg to about 12 mg of active pharmacological agent, from about 13 mg to about 19 mg of active pharmacological agent, from about 20 mg to about 30 mg of active pharmacological agent, from about 31 mg to about 60 mg of active pharmacological agent, from about 61 mg to about 80 mg of active pharmacological agent, and from about 81 mg to about 110 mg of active pharmacological agent. One embodiment is a 500 mg capsule containing 100 mg of pharmacologically active agent (i.e. 20% by weight of the pharmaceutical composition).

In one embodiment, the invention provides a process for preparing a preferred pharmaceutical composition comprising:

a) 20% by weight of the composition of the active pharmacological agent 4-(3-{5-chloro-1-(diphenylmethyl)-2-[2-({[2(trifluoromethyl)benzyl]sulfonyl}amino)ethyl]-1H-indol-3-yl}propyl)benzoic acid or a pharmaceutically acceptable salt thereof; and

b) a carrier or excipient system comprising:
i) PEG 1000 in an amount of about 20% by weight of the composition;
ii) polysorbate 80 in an amount of about 10% by weight of the composition;
iii) PEG 400 in an amount of about 40% by weight of the composition; and
iv) PVP K-17 in an amount of about 10% by weight of the composition;
   said process comprising

(1) mixing the PEG 1000, polysorbate 80, and PEG 400 to produce a first homogenous solution;

(2) slowly adding the PVP K-17 until dissolved to form a second homogenous solution;

(3) slowly adding the pharmacologically active agent to the second homogenous solution;

(4) mixing with sufficient heating until the pharmacologically active agent is dissolved to produce a third homogenous solution.

As with the other embodiments described herein, the process can further comprise one or more of the following additional steps:
heating the PEG 1000, polysorbate 80, and PEG 400 to a temperature sufficient to produce a first homogenous solution (e.g., from about 90° C. to about 100° C.);
cooling the second homogenous solution (e.g., to about 80° C. to about 90° C.) prior to slowly adding the pharmacologically active agent to the second homogenous solution;
encapsulating at least a portion of the third homogenous solution into one or more unit dosage capsule forms; and
cooling the third homogenous solution (e.g., to about 40° C.) prior to encapsulation.

The invention further includes any product made by any of the processes described herein.

As used herein, the terms “pharmaceutically effective amount” or “therapeutically effective amount” mean the total amount of each active component of the pharmaceutical composition or method that is sufficient to show a meaningful patient benefit, i.e., treatment, healing, prevention, inhibition or amelioration of a physiological response or condition, such as an inflammatory condition or pain, or an increase in rate of treatment, healing, prevention, inhibition or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

The term “pharmaceutically acceptable” means a non-toxic material that does not interfere with the effectiveness of the biological activity of the active ingredient(s).

The term “% by weight of the composition” and the weight percentages set forth for each of the components of the compositions disclosed herein refer to the percentages that each component will comprise in a final pharmaceutical composition based on the weight of the composition, excluding any surface covering, such as a tablet coating or encapsulating material, such as a capsule.

GELUCIRE as used herein refers to a family of vehicles derived from mixtures of mono-, di-, and triglycerides with polyethylene glycol (PEG) esters of fatty acids. Such as a mixture of glycerol and PEG1500 esters of long fatty acids. Gelucires are available with a range of properties depending on their Hydrophilic Lipophilic Balance (HLB 1-18) and melting point (33° C.-65° C.) range. The suffixes refer respectively to its melting point and its HLB. Gelucire 44/14 and Gelucire 50/13 are examples of such compounds, available from Gattefosse.

As will be appreciated, some components of the formulations of the invention can possess multiple functions. For example, a given component can act as both a diluent and a solubilizer. In some such cases, the function of a given component can be considered singular, even though its properties may allow multiple functionality.

The pharmaceutical formulations and excipient systems herein can also contain an antioxidant or a mixture of antioxidants, such as ascorbic acid. Other antioxidants, which can be used, include sodium ascorbate and ascorbyl palmitate, optionally in conjunction with an amount of ascorbic acid. An example range for the antioxidant(s) is from about up to about 15% by weight, e.g., from about 0.05% to about 15% by weight, from about 0.5% to about 15% by weight, or from about 0.5% to about 5% by weight. In some embodiments, the pharmaceutical formulations contain substantially no antioxidant.

Additional numerous various viscosity builders, solubilizers, diluents, stabilizers, excipients, dosage forms, and the like, that are suitable for use in connection with the pharmaceutical compositions of the invention are known in the art and described in, for example, Remington: The Science and Practice of Pharmacy, 20th edition, Alfonoso R. Gennaro (ed.), Lippincott Williams & Wilkins, Baltimore, Md. (2000), which is incorporated herein by reference in its entirety.

The materials, methods, and examples presented herein are intended to be illustrative, and are not intended to limit the scope of the invention. All publications, patent applications, patents, and other references mentioned herein are incorporated by reference in their entirety.

### EXAMPLES

### A. Preparation of Compounds of Formula I or Formula II

The compounds of Formula I and II can be conveniently prepared in accordance with the procedures outlined in the schemes below, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but one skilled in the art can determine such conditions by routine optimization procedures. Those skilled in the art will recognize that the nature and order of the synthetic steps presented may be varied for the purpose of optimizing the formation of the compounds of the invention.

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 4th Ed., Wiley & Sons, 2006, which is incorporated herein by reference in its entirety.

Examples of compounds of Formula I or Formula II and methods for synthesizing them can be found in U.S. Pat. Nos. 6,797,708; 6,891,065 and 6,984,735 and U.S. patent application Ser. Nos. 10/930,534 (filed Aug. 31, 2004), 10/948,004 (filed Sep. 23, 2004), 10/989,840 (filed Nov. 16, 2004), 11/014,657 (filed Dec. 16, 2004), 11/064,241 (filed Feb. 23, 2005), 11/088,568 (filed Mar. 24, 2005), 11/140,390 (filed May 27, 2005), 11/207,072 (filed Aug. 18, 2005) and 11/442,199 (filed May 26, 2006), each of which is incorporated by reference in their entireties.

Examples of compounds of Formula I and Formula II include, but are not limited to:

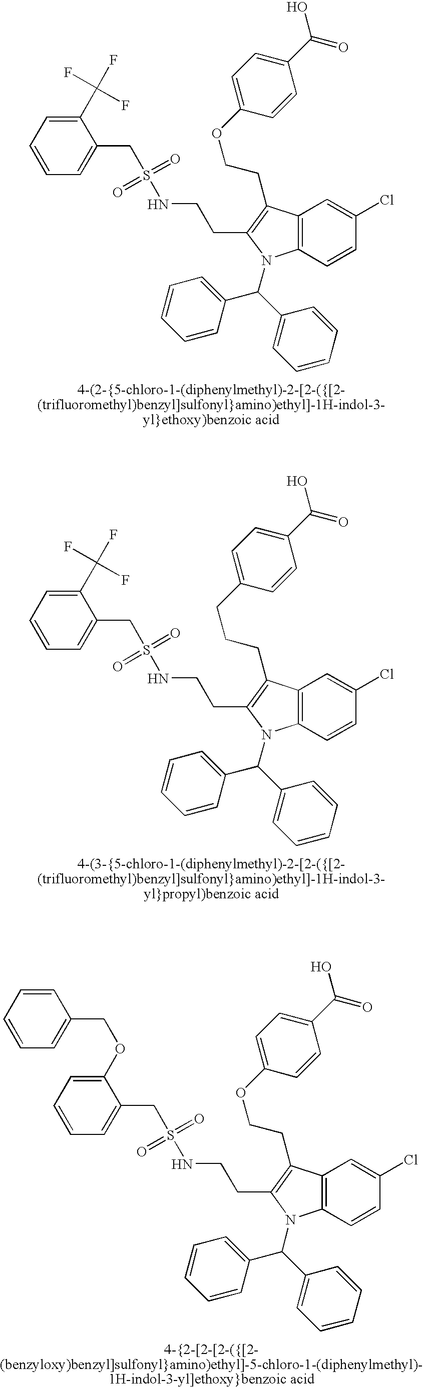

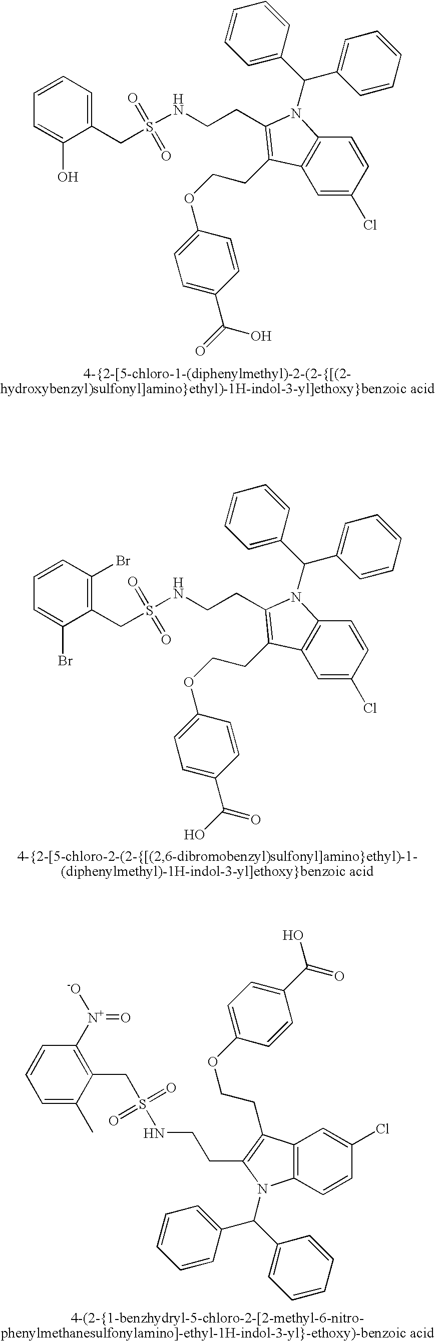

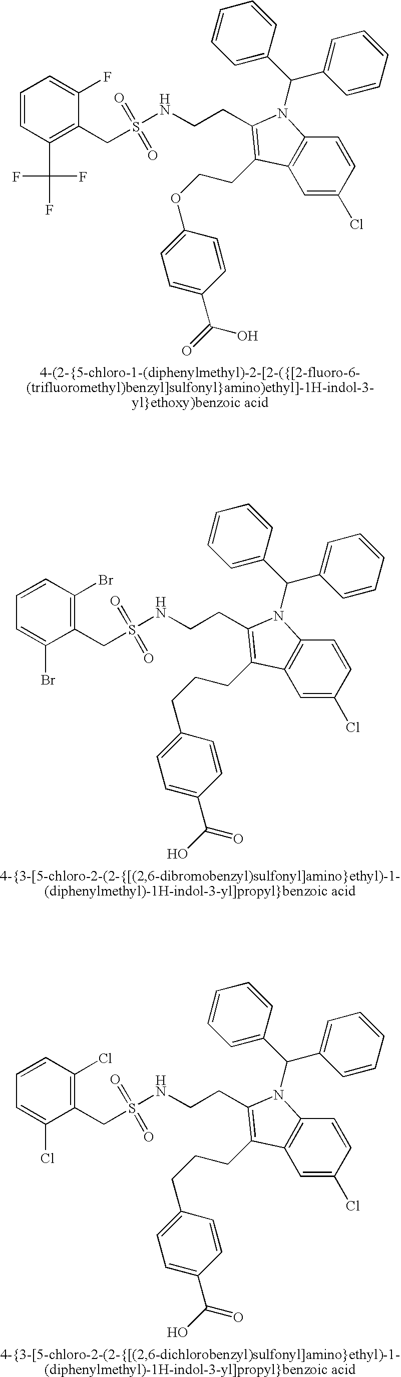

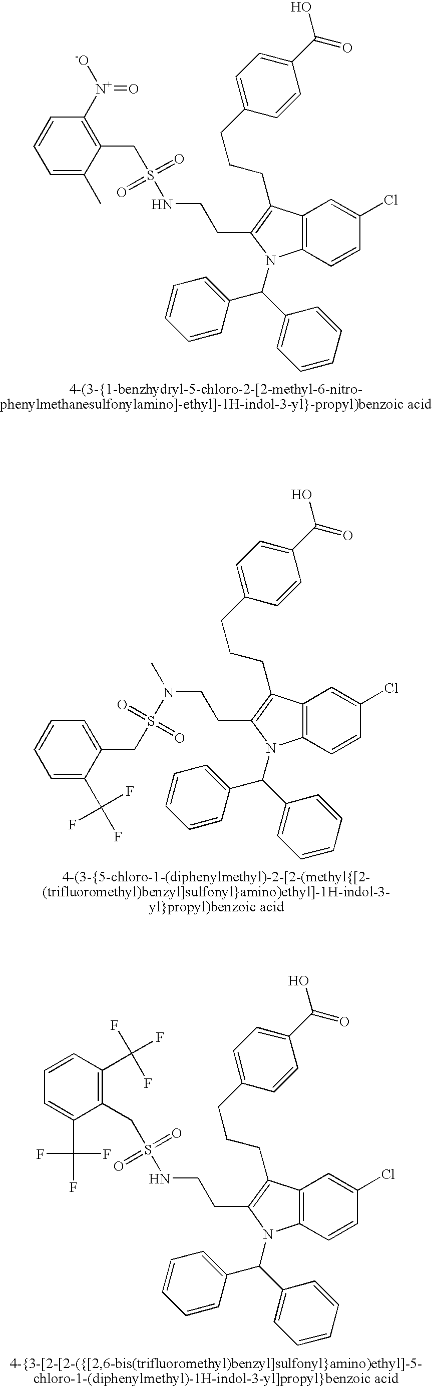

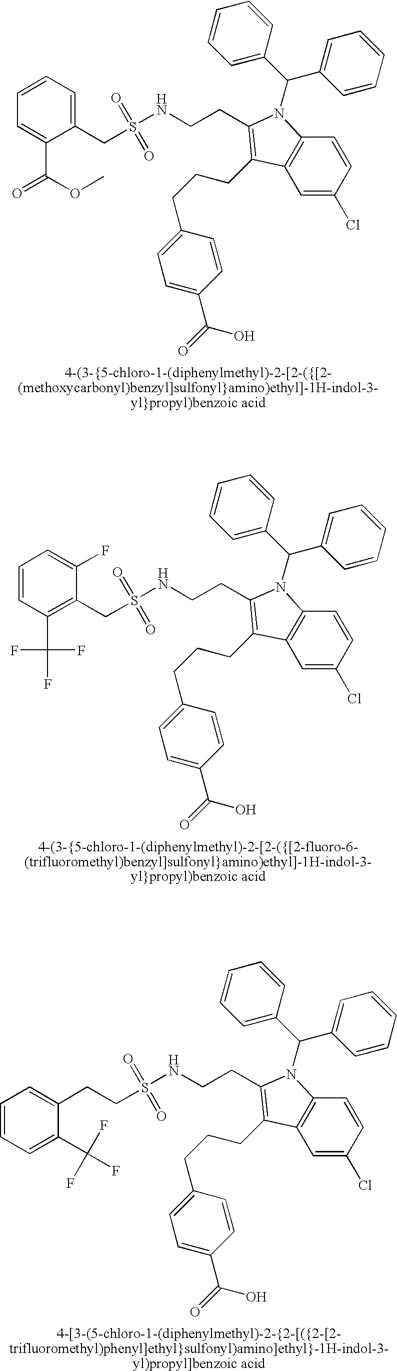

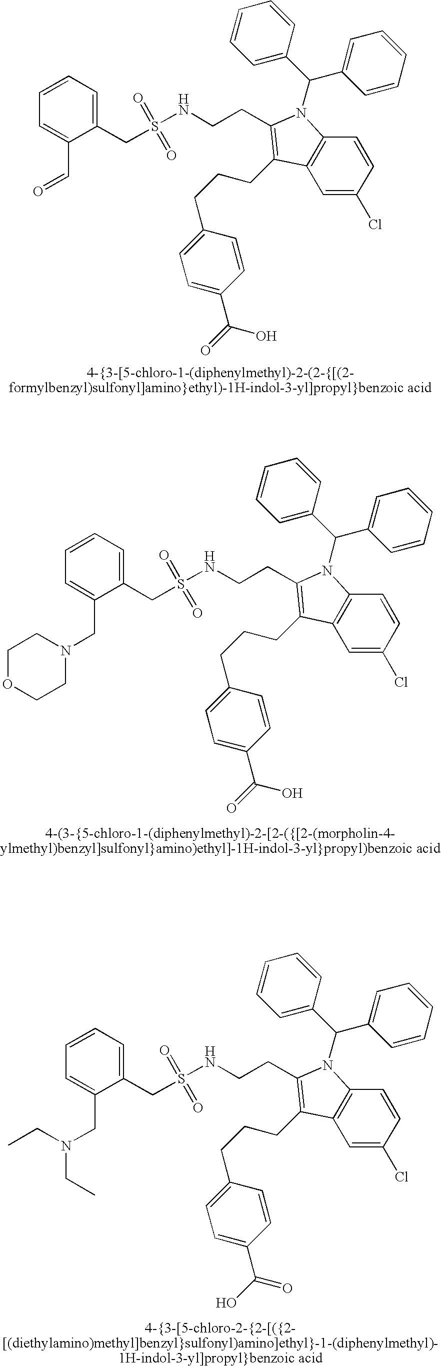

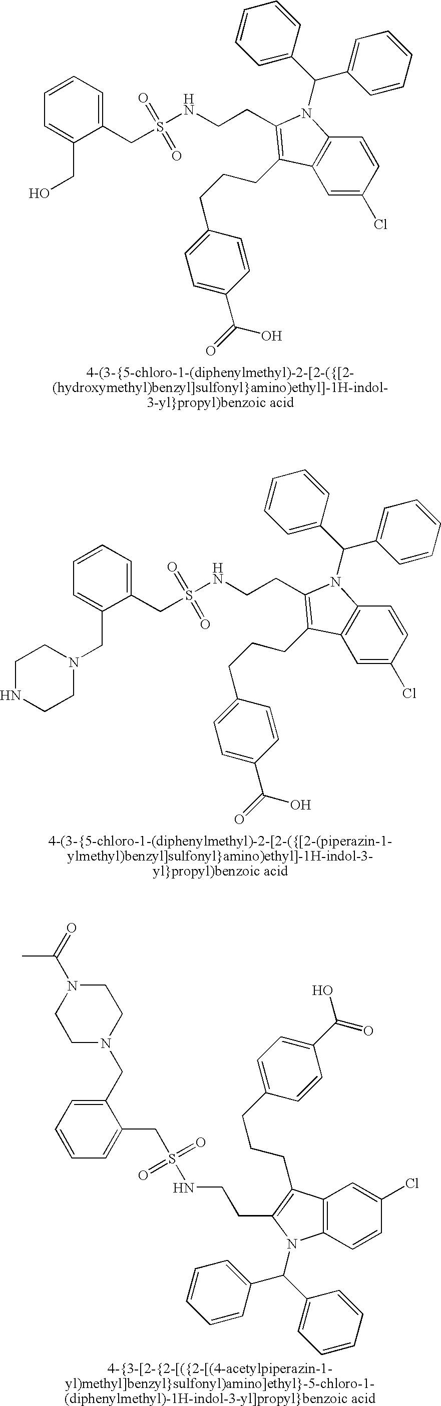

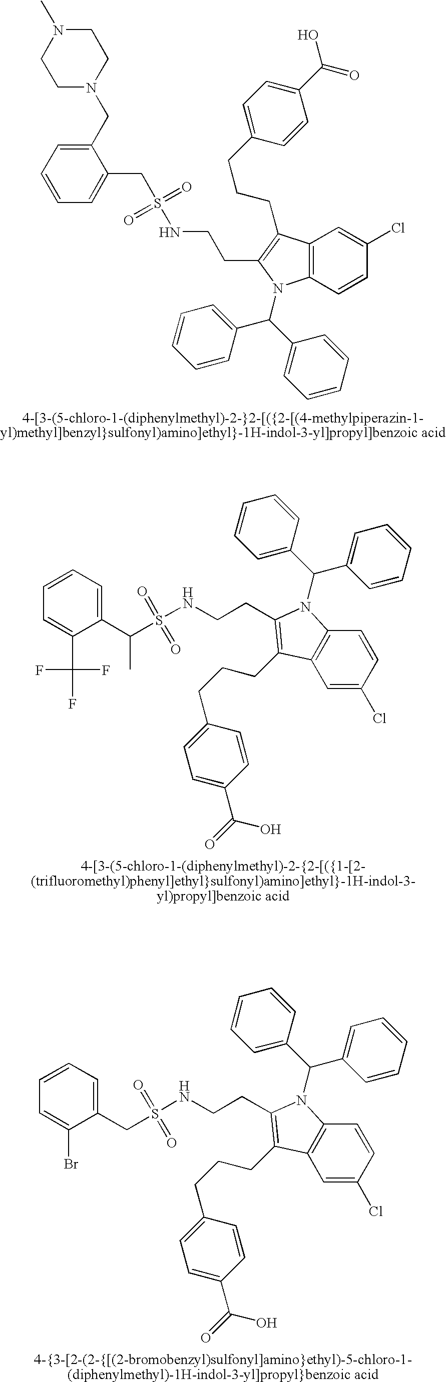

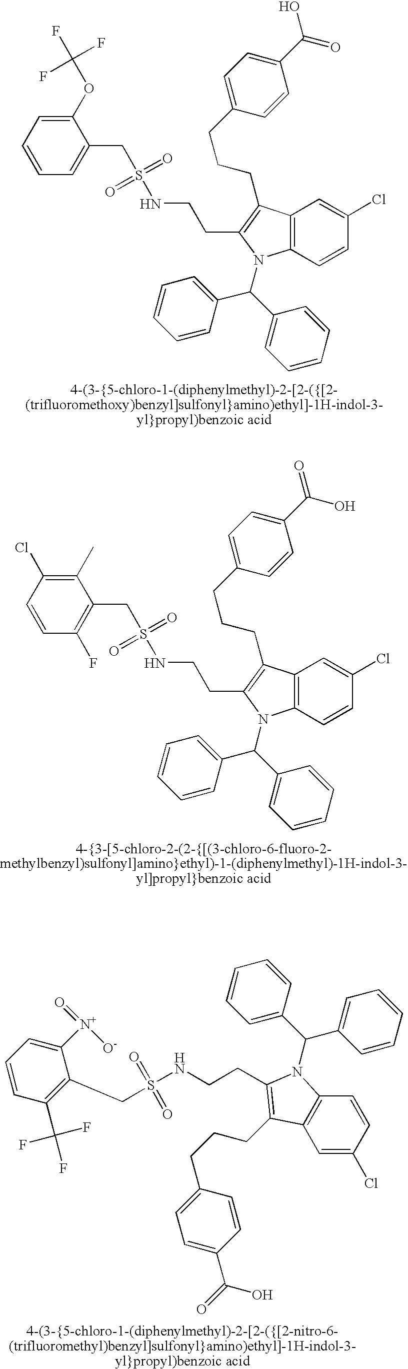

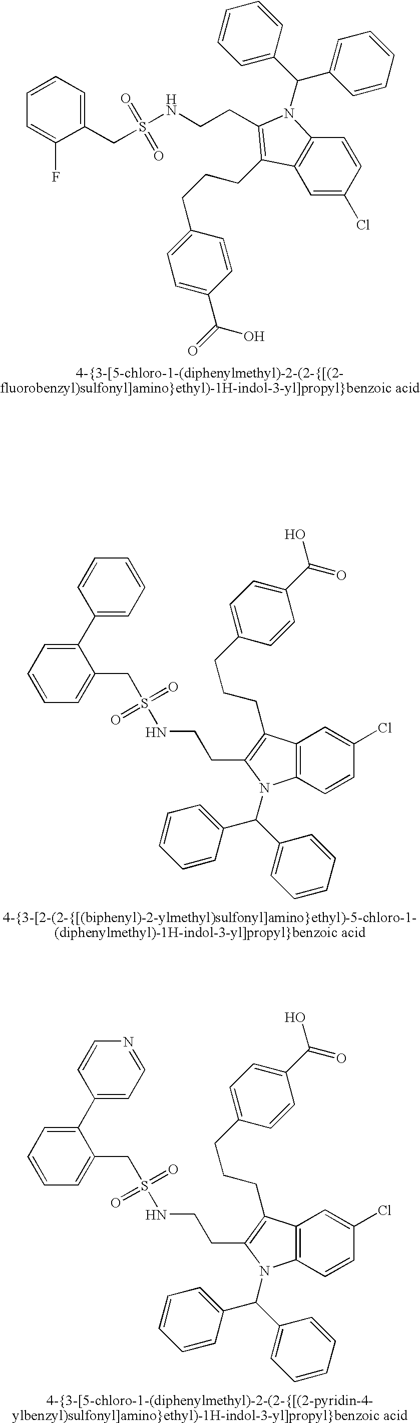

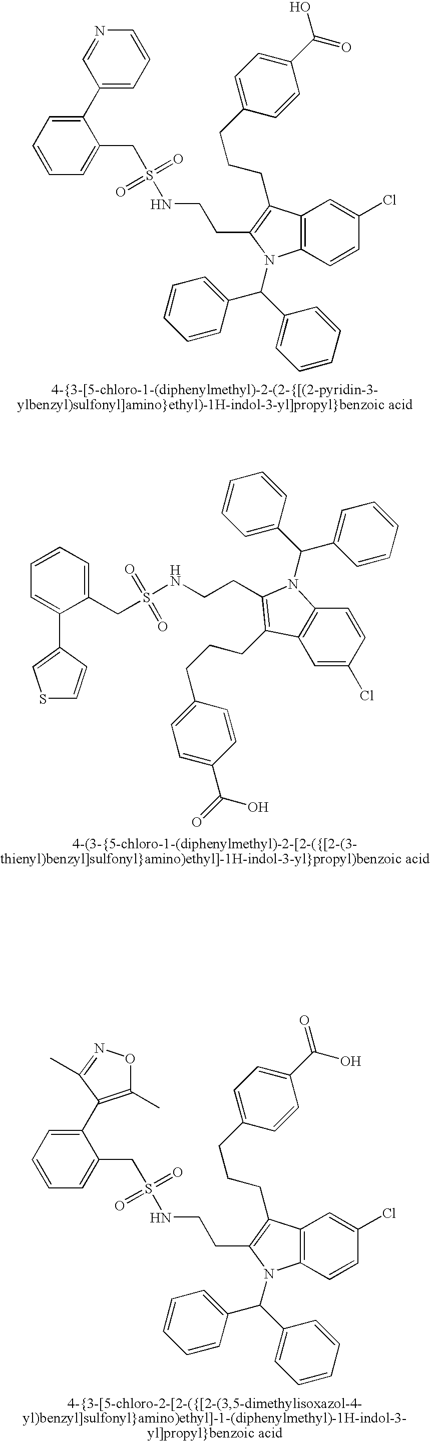

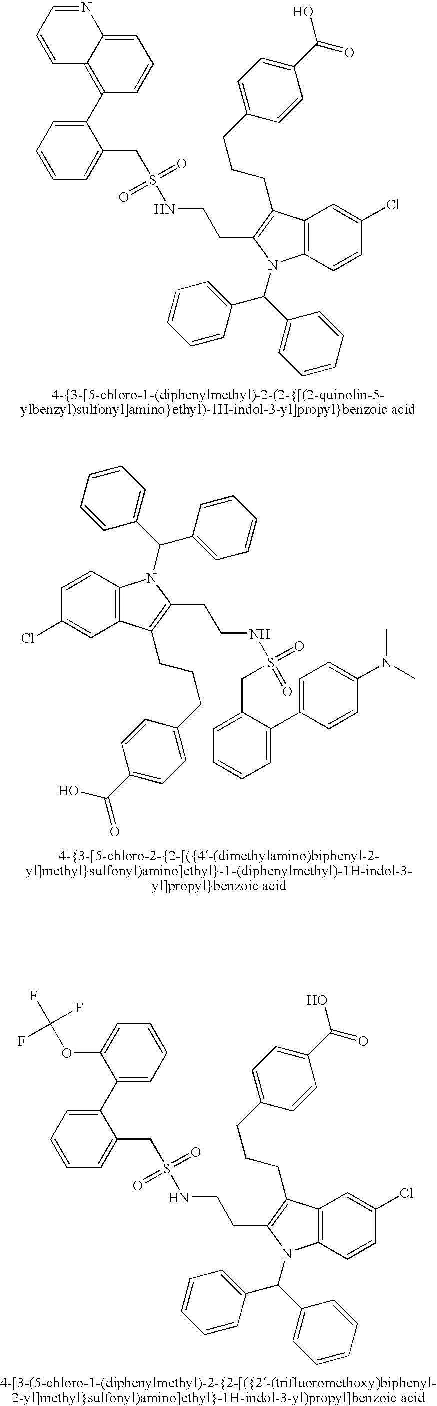

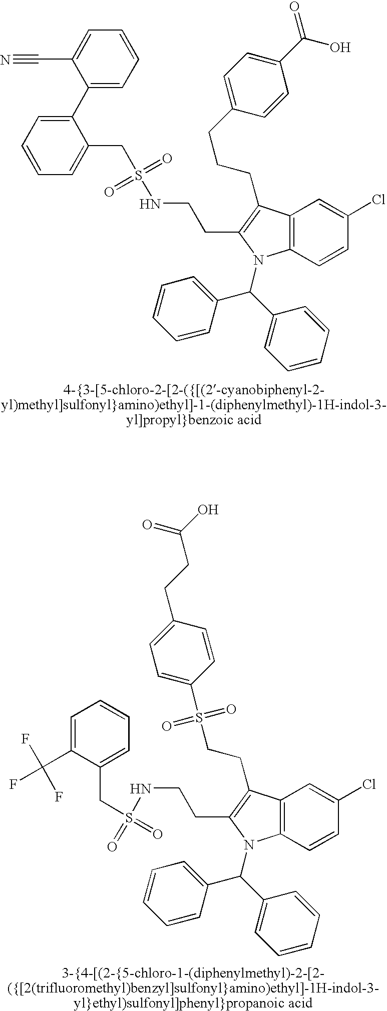

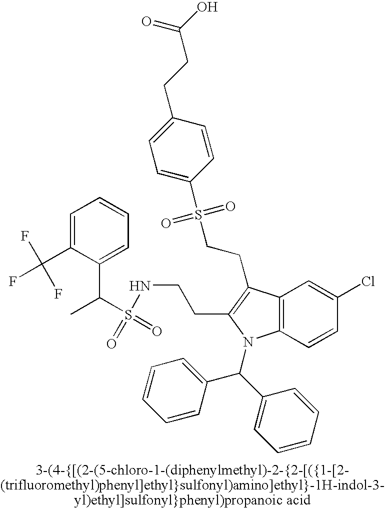

### B. Preparation of 100 Mg Dose Capsule

A 500 mg unit dosage capsule in accordance with the invention, containing a 100 mg dose of 4-(3-{5-chloro-1-(diphenylmethyl)-2-[2-({[2-(trifluoromethyl)benzyl]sulfonyl}amino) ethyl]-1H-indol-3-yl}propyl)benzoic acid was prepared as described in Table 1.

TABLE 1

| | | % Wt of- | |
|---|---|---|---|
| | | Compo- | Weight |
| Component | Compound | sition | (mg) |
| Pharmacological | 4-(3-{5-chloro-1- | 20 | 100 |
| Agent | (diphenylmethyl)-2-[2-({[2- | | |
| | (trifluoromethyl)benzyl]- | | |
| | sulfonyl}amino)ethyl]- | | |
| | 1H-indol-3- | | |
| | yl}propyl)benzoic acid | | |
| Viscosity Modifier | PEG 1000 | 20 | 100 |
| Solubilizer | polysorbate 80 | 10 | 50 |
| Diluent | PEG 400 | 40 | 200 |
| Stabilizer | PVP-K-17 | 10 | 50 |

The pharmaceutical composition described above was prepared for administration via a capsule as follows:
1. PEG 1000 (7.5 g), PEG 400 (20 g), Polysorbate 80 (5 g) were added to an appropriate mixing vessel equipped for temperature control.
2. The vessel was heated to 95 +/−5° C. with mixing until a homogeneous solution was obtained.
3. The PVP K-17 (5 g) was added slowly until dissolved.
4. The vessel was cooled to 85 +/−5° C.
5. 4-(3-{5-chloro-1-(diphenylmethyl)-2-[2-({[2-(trifluoromethyl)benzyl]sulfonyl}amino)ethyl]-1H-indol-3-yl}propyl)benzoic acid (5 g) was added slowly into the solution from Step 4 with mixing at 85 +/−5° C. until the drug was dissolved and a homogeneous solution was obtained.
6. The resultant solution was cooled to 40 +/−5° C. with mixing.
7. 0.500 g of the finished solution from Step 6 was encapsulated into size #0 capsules.

Any suitable encapsulating techniques and apparatus may be used. The resultant capsule is approximately a 500 mg capsule, which delivers approximately 100 mg of the pharmacological agent. Other suitable doses and capsule sizes can be made in accordance with the disclosure herein. In particular, those of skill in the art, will readily recognize that 10, 25, 50 and 75 mg unit dosage forms, and others, can be made through similar methods.

### C. Dissolution Testing

The solubility of 4-(3-{5-chloro-1-(diphenylmethyl)-2-[2-({[2-(trifluoromethyl)benzyl]sulfonyl}amino)ethyl]-1H-indol-3-yl}propyl)benzoic acid was measured at room temperature in water, acid and basic conditions. The intrinsic solubility of the free acid was below the HPLC detection limit of 31 ng/mL, whereas the anion had a solubility of 110 ng/mL.

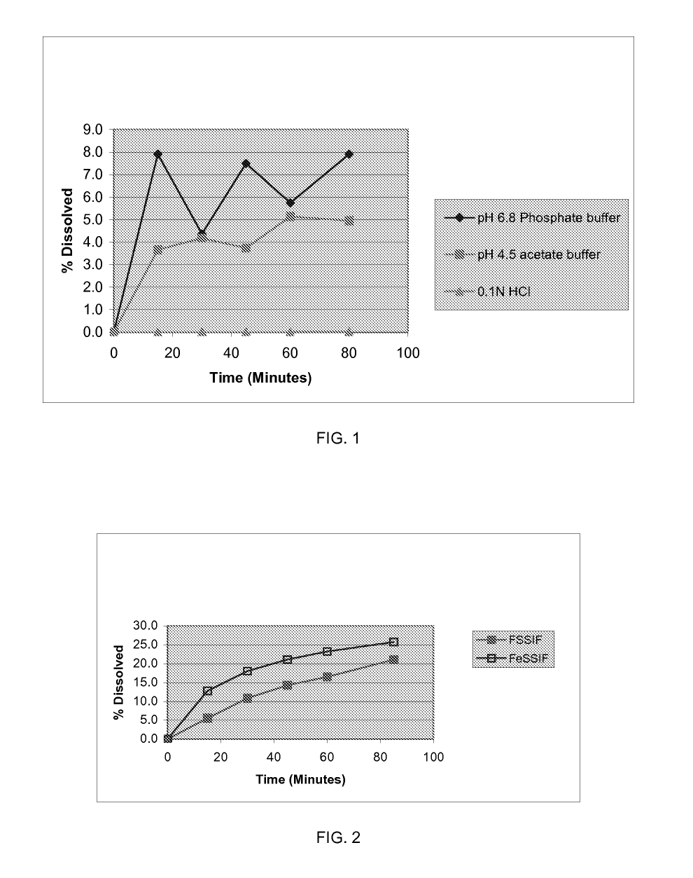

Dissolution testing was performed on 100 mg strength capsules produced according to the procedure described above. Capsules were placed in 900 mL of aqueous solutions having pH 1 (0.1 N HCl), pH 6.8 (50 mM sodium phosphate buffer) and pH 4.5 (mM sodium acetate buffer). The UV absorption of each solution was measured at various timepoints (1 mm path length, 237 nm) and the percent dissolution was calculated compared to a standard response at that wavelength. As shown in FIG. 1, at pH 1 there was practically no dissolution, while at pH 4.5 and 6.8 the capsule was slightly more soluble.

Dissolution testing was then performed on 100 mg strength capsules produced according to the procedure described above in Fasted State Simulated Intestinal Fluid (FSSIF: 0.029 M KH₂PO₄, 5 mM sodium taurocholate, 1.5 mM lecithin, 0.22 M KCl, pH adjusted to 6.8 with NaOH) and Fed State Simulated Intestinal Fluid (FeSSIF: 0.144 M acetic acid, 15 mM sodium taurocholate, 4 mM lecithin, 0.19 M KCl, pH adjusted to 5.0 with NaOH) to simulate fed and fasted conditions in the gut. As shown in FIG. 2, there was an appreciable increase in dissolution rate in the simulated fed and fasted media when compared to the previous results, with an increase in dissolution in the simulated fed media.

### D. In Vivo Dog Exposure Studies

A formulation containing 4-(3-{5-chloro-1-(diphenylmethyl)-2-[2-({[2-(trifluoromethyl)benzyl]sulfonyl}amino)ethyl]-1H-indol-3-yl}propyl)benzoic acid according to the invention was studied in dogs in a high fat-fed/fasted study at approximately 12 mg/kg. To simulate the fed state, three female beagle dogs were fed a high-fat diet by oral gavage 30 minutes prior to dosing with 100 mg dose capsules as described in Table 1 above. Blood samples were drawn at 0, 0.5, 1, 2, 3, 4, 6, 8, 12 and 24 hours. The dogs were then fed ⅔ of the daily food ration after the 4 hour blood draw. Blood samples were stored on ice, centrifuged at 5° C., and the plasma was collected and stored at −70° C. The plasma samples were analyzed by LC/MS/MS to determine the amount of 4-(3-{5-chloro-1-(diphenylmethyl)-2-[2-({[2-(trifluoromethyl)benzyl]sulfonyl}amino)ethyl]-1H-indol-3-yl}propyl)benzoic acid in the sample.

To simulate the fasted state, the above procedure was repeated with the same three female beagle dogs that were fasted overnight prior to dosing, then fed after the 4 hour blood draw. The results of both the fed and fasted studies are summarized in Table 2 (reported results are the average of the data from the three test animals).

TABLE 2

| | Cₘₐₓ | AUC_{inf} | | | % Bio- | Fed/Fasted | Fed/Fasted |
|---|---|---|---|---|---|---|---|
| Formulation | (ng/mL) | (ng hr/mL) | AUC/Dose | Cₘₐₓ/Dose | availability | AUC/Dose | Cₘₐₓ/Dose |
| Fasted | 1069 | 9988 | 957 | 100.8 | 5.04 | 1.92 | 2.95 |
| Fed | 20049 | 20049 | 1964 | 321.5 | 10.34 | | |

Data from a rat carrageenan-induced paw edema (CPE) study indicated the minimum efficacious exposure of 4-(3-{5-chloro-1-(diphenylmethyl)-2-[2-({[2-(trifluoromethyl)benzyl]sulfonyl}amino) ethyl]-1H-indol-3-yl}propyl)benzoic acid was 1360 ng*hr/ml. The data in Table 2 shows that the formulation according to the present invention results in an exposure of about 7 times the efficacious exposure in the fasted state and about 15 times the efficacious exposure in the fed state. These exposures translate into percent bioavailabilities of 5.0 and 10.3 when compared to an IV formulation (15% 4-(3-{5-chloro-1-(diphenylmethyl)-2-[2-({[2-(trifluoromethyl)benzyl]sulfonyl}amino)ethyl]-1H-indol-3-yl}propyl)benzoic acid, 10% EtOH, 75% Solutol HS-15, diluted to 2 mg/mL with sterile water for injection).

All publications mentioned herein, including but not limited to patent applications, patents, and other references, are incorporated by reference in their entirety.

The materials, methods, and examples presented herein are intended to be illustrative, and are not intended to limit the scope of the invention.

1. A pharmaceutical composition comprising
a) a pharmaceutically effective amount of an active pharmacological agent having Formula I: 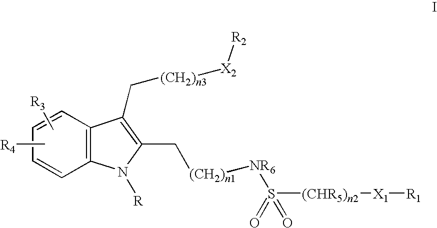
or a pharmaceutically acceptable salt thereof, wherein:
R is selected from the formulae —(CH₂)ₙ-A, —(CH₂)ₙ—S-A, and —(CH₂)ₙ—O-A, wherein A is selected from the moieties: 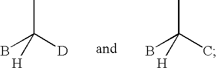
wherein
D is C₁-C₆ alkyl, C₁-C₆ alkoxy, C₃-C₆ cycloalkyl, —CF₃, or —(CH₂)₁₋₃—CF₃;
B and C are independently selected from phenyl, pyridinyl, pyrimidinyl, furyl, thienyl and pyrrolyl groups, each optionally substituted by from 1 to 3 substituents selected independently from halogen, —CN, —CHO, —CF₃, —OCF₃, —OH, —C₁-C₆ alkyl, C₁-C₆ alkoxy, —NH₂, —N(C₁-C₆ alkyl)₂, —NH(C₁-C₆ alkyl), —NH—C(O)—(C₁-C₆ alkyl), —NO₂, or by a 5- or 6-membered heterocyclic or heteroaromatic ring containing 1 or 2 heteroatoms selected from O, N and S;
n is an integer from 0 to 3;
n₁ is an integer from 1 to 3;
n₂ is an integer from 0 to 4;
n₃ is an integer from 0 to 3;
n₄ is an integer from 0 to 2;
X₁ is selected from a chemical bond, —S—, —O—, —S(O)—, —S(O)₂—, —NH—, —C═C— 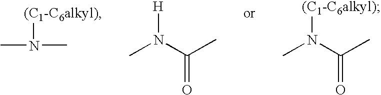
R₁ is selected from C₁-C₆ alkyl, C₁-C₆ fluorinated alkyl, C₃-C₆ cycloalkyl, tetrahydropyranyl, camphoryl, adamantyl, CN, —N(C₁-C₆ alkyl)₂, phenyl, pyridinyl, pyrimidinyl, furyl, thienyl, naphthyl, morpholinyl, triazolyl, pyrazolyl, piperidinyl, pyrrolidinyl, imidazolyl, piperizinyl, thiazolidinyl, thiomorpholinyl, tetrazolyl, indolyl, benzoxazolyl, benzofuranyl, imidazolidine-2-thionyl, 7,7-dimethyl-bicyclo[2.2.1]heptan-2-onyl, benzo[1,2,5]oxadiazolyl, 2-oxa-5-aza-bicyclo[2.2.1]heptanyl, piperazin-2-onyl and pyrrolyl groups, each optionally substituted by from 1 to 3 substituents independently selected from halogen, —CN, —CHO, —CF₃, —OCF₃, —OH, C₁-C₆ alkyl, C₁-C₆ alkoxy, —NH₂, —N(C₁-C₆ alkyl)₂, —NH(C₁-C₆ alkyl), —NH—C(O)—(C₁-C₆ alkyl), —NO₂, —SO₂(C₁-C₃ alkyl), —SO₂NH₂, —SO₂NH(C₁-C₃ alkyl), —SO₂N(C₁-C₃ alkyl)₂, —COOH, —CH₂—COOH, —CH₂—NH(C₁-C₆ alkyl), —CH₂—N(C₁-C₆ alkyl)₂, —CH₂—NH₂, pyridinyl, 2-methyl-thiazolyl, morpholino, 1-chloro-2-methyl-propyl, C₁-C₆thioalkyl, phenyl (further optionally substituted with one or more halogens, dialkylamino, —CN, or —OCF₃), benzyloxy, —(C₁-C₃ alkyl)C(O)CH₃, —(C₁-C₃ alkyl)OCH₃, —C(O)NH₂, or 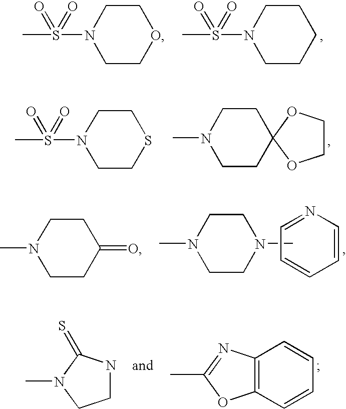
X₂ is selected from —O—, —CH₂—, —S—, —SO—, —SO₂—, —NH—, —C(O)—, 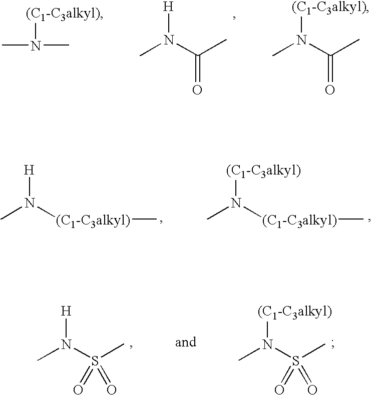
R₂ is a ring moiety selected from phenyl, pyridinyl, pyrimidinyl, furyl, thienyl and pyrrolyl groups, the ring moiety being substituted by a group of the formula —(CH₂)ₙ₄—CO₂H or a pharmaceutically acceptable acid mimic or mimetic; and also optionally substituted by 1 or 2 additional substituents independently selected from halogen, —CN, —CHO, —CF₃, —OCF₃, —OH, —C₁-C₆ alkyl, C₁-C₆ alkoxy, C₁-C₆ thioalkyl, —NH₂, —N(C₁-C₆ alkyl)₂, —NH(C₁-C₆ alkyl), —NH—C(O)—(C₁-C₆ alkyl), and —NO₂;
R₃ is selected from H, halogen, —CN, —CHO, —CF₃, —OCF₃, —OH, C₁-C₆ alkyl, C₁-C₆ alkoxy, C₁-C₆ thioalkyl, —NH₂, —N(C₁-C₆ alkyl)₂, —NH(C₁-C₆ alkyl), —NH—C(O)—(C₁-C₆ alkyl), and —NO₂;
R₄ is selected from H, halogen, —CN, —CHO, —CF₃, —OCF₃, —OH, C₁-C₆ alkyl, C₁-C₆ alkoxy, C₁-C₆ thioalkyl, —NH₂, —N(C₁-C₆ alkyl)₂, —NH(C₁-C₆ alkyl), —NH—C(O)—(C₁-C₆ alkyl), —NO₂, —NH—C(O)—N(C₁-C₃ alkyl)₂, —NH—C(O)—NH(C₁-C₃ alkyl), —NH—C(O)—O—(C₁-C₃ alkyl), —SO₂—C₁-C₆ alkyl, —S—C₃-C₆ cycloalkyl, —S—CH₂—C₃-C₆ cycloalkyl, —SO₂—C₃-C₆ cycloalkyl, —SO₂—CH₂—C₃-C₆ cycloalkyl, C₃-C₆ cycloalkyl, —CH₂—C₃-C₆ cycloalkyl, —O—C₃-C₆ cycloalkyl, —O—CH₂—C₃-C₆ cycloalkyl, phenyl, benzyl, benzyloxy, morpholino, pyrrolidino, piperidinyl, piperizinyl, furanyl, thienyl, imidazolyl, tetrazolyl, pyrazinyl, pyrazolonyl, pyrazolyl, oxazolyl, and isoxazolyl, the rings of each of these R₄ groups each being optionally substituted by from 1 to 3 substituents selected from the group of halogen, —CN, —CHO, —CF₃, —OH, C₁-C₆ alkyl, C₁-C₆ alkoxy, —NH₂, —N(C₁-C₆ alkyl)₂, —NH(C₁-C₆ alkyl), —NH—C(O)—(C₁-C₆ alkyl), —NO₂, —SO₂(C₁-C₃ alkyl), —SO₂NH(C₁-C₃ alkyl), —SO₂N(C₁-C₃ alkyl)₂, and —OCF₃;
each R₅ is independently H or C1-3 alkyl; and
R₆ is H or C₁₋₆ alkyl; and
b) a carrier or excipient system comprising:
i) about 15 to about 25% a viscosity builder by weight of the composition;
(ii) about 5 to about 15% a solubilizer by weight of the composition; and
iii) about 10 to about 50% a diluent by weight of the composition; and
iv) about 1 to about 10% a stabilizer by weight of the composition.

2. The pharmaceutical composition of claim 1, wherein
R₁ is optionally substituted phenyl; and
R is 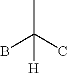
where B and C are phenyl.

3. The pharmaceutical composition of claim 1, wherein said pharmaceutically effective amount of said active pharmacological agent is about 0.1 to about 25% by weight of the composition.

4. The pharmaceutical composition of claim 1, wherein said viscosity builder is selected from the group consisting of PEG 1000, PEG 1500, Gelucire 44/14, Gelucire 50/13, and mixtures thereof.

5. The pharmaceutical composition of claim 1, wherein said viscosity builder comprises PEG 1000.

6. The pharmaceutical composition of claim 1 wherein said solubilizer is selected from the group consisting of polysorbate 80, polyoxyl 40 hydrogenated castor oil, polyoxyl 35 castor oil, and mixtures thereof.

7. The pharmaceutical composition of claim 1, wherein said solubilizer comprises polysorbate 80.

8. The pharmaceutical composition of claim 1, wherein said diluent is selected from the group consisting of PEG 400, propylene glycol, propylene carbonate, triacetin, and mixtures thereof.

9. The pharmaceutical composition of claim 1, wherein said diluent comprises PEG 400.

10. The pharmaceutical composition of claim 1, wherein said stabilizer is a polyvinylpyrrolidone.

11. The pharmaceutical composition of claim 1, wherein said stabilizer is selected from polyvinylpyrrolidone 12, polyvinylpyrrolidone 17 and mixtures thereof.

12. The pharmaceutical composition of claim 1, wherein said carrier or excipient system comprises:
i) said viscosity builder is selected from the group consisting of PEG 1000, PEG 1500, Gelucire 44/14, Gelucire 50/13, and mixtures thereof;
ii) said solubilizer is selected from the group consisting of polysorbate 80, polyoxyl 40 hydrogenated castor oil, polyoxyl 35 castor oil, and mixtures thereof;
iii) said diluent is selected from the group consisting of PEG 400, propylene glycol, propylene carbonate. Triacetin, and mixtures thereof; and
iv) said stabilizer is a polyvinylpyrrolidone.

13. The pharmaceutical composition of claim 1, wherein said carrier or excipient system comprises:
i) PEG 1000 in an amount of from about 15% to about 25% by weight of the composition;
ii) polysorbate 80 in an amount of from about 5% to about 15% by weight of the composition;
iii) PEG 400 in an amount of from about 10% to about 50% by weight of the composition; and
iv) PVP K-17 in an amount of from about 1% to about 10% by weight of the composition.

14. A pharmaceutical composition comprising:
a) a pharmaceutically effective amount of an active pharmacological agent having the Formula II: 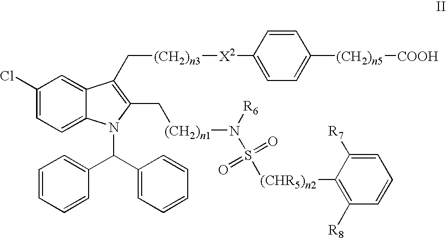
or a pharmaceutically acceptable salt thereof, wherein:
n₁ is 1 or 2;
n₂ is 1 or 2;
n₃ is 1 or 2;
n₅ is 0, 1 or 2;
X² is O, —CH₂— or SO₂;
each R₅ is independently H or C₁₋₃ alkyl;
R₆ is H or C₁₋₆ alkyl;
R₇ is selected from the group consisting of —OH, benzyloxy, —CH₃, —CF₃, —OCF₃, C₁₋₃ alkoxy, halogen, —CHO, —CO(C₁₋₃ alkyl), —CO(OC₁₋₃ alkyl), quinoline-5-yl, 3,5-dimethylisoxazol-4-yl, thiophene-3-yl, pyridin-4-yl, pyridine-3-yl, —CH₂-Q, and phenyl optionally substituted by from one to three independently selected R₃₀ groups;
R₈ is selected from the group consisting of H, —OH, —NO₂, —CF₃, —OCF₃, C₁₋₃ alkoxy, halogen, —CO(C₁₋₃ alkyl), —CO(OC₁₋₃ alkyl), quinoline-5-yl, 3,5-dimethylisoxazol-4-yl, thiophene-3-yl, —CH₂-Q, and phenyl substituted by from one to three independently selected R₃₀ groups;
Q is OH, dialkylamino, 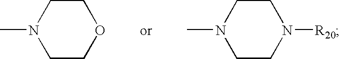
R₂₀ is selected from the group consisting of H, C₁₋₃ alkyl, and —CO(C₁₋₃ alkyl); and
R₃₀ is selected from the group consisting of dialkylamino, —CN and —OCF₃; provided that:
i) when each R₅ is H, R₆ is H, n₅ is 0, and R₈ is H, then R₇ cannot be chlorine;
ii) when each R₅ is H, R₆ is H, n₅ is 0, X² is O or —CH₂—, and R₈ is H, then R₇ cannot be CH₃;
iii) when each R₅ is H, and R₆ is H, then R₇ and R₈ cannot both be fluorine;
iv) when each R₅ is H, R₆ is H, and X² is O, then R₇ and R₈ cannot both be chlorine;
v) when each R₅ is H, R₆ is H, X² is O, and R₈ is NO₂, then R₇ cannot be fluorine; and
vi) when each R₅ is H, R₆ is H, X² is SO₂, and R₈ is H, then R₇ cannot be fluorine or chlorine; and
b) a carrier or excipient system comprising:
i) about 15 to about 25% a viscosity builder by weight of the composition;
about 5 to about 15% a solubilizer by weight of the composition; and
(iii) about 10 to about 50% a diluent by weight of the composition; and
iv) about 1 to about 10% a stabilizer by weight of the composition.

15. The pharmaceutical composition of claim 14, wherein the compound of Formula II has the Formula III: 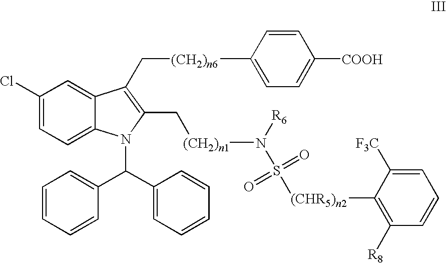
or a pharmaceutically acceptable salt thereof, wherein:
n₁ is 1 or 2;
n₂ is 1 or 2;
n₆ is 1 or 2;
R₅ is H or CH₃;
R₆ is H or C₁₋₆ alkyl; and
R₈ is selected from the group consisting of H, —OH, —NO₂, —CF₃, —OCF₃, —OCH₃, halogen, —COCH₃, —COOCH₃, dimethylamino, diethylamino and —CN.

16. The pharmaceutical composition of claim 14, wherein the compound of Formula II is 4-(3-{5-chloro-1-(diphenylmethyl)-2-[2-({[2-(trifluoromethyl)benzyl]sulfonyl}amino)ethyl]-1H-indol-3-yl}propyl)benzoic acid or a pharmaceutically acceptable salt thereof.

17. The pharmaceutical composition of claim 14, wherein said pharmaceutically effective amount of said active pharmacological agent is about 0.1 to about 25% by weight of the composition.

18. The pharmaceutical composition of claim 14, wherein said viscosity builder is selected from the group consisting of PEG 1000, PEG 1500, Gelucire 44/14, Gelucire 50/13, and mixtures thereof.

19. The pharmaceutical composition of claim 14, wherein said viscosity builder comprises PEG 1000.

20. The pharmaceutical composition of claim 14 wherein said solubilizer is selected from the group consisting of polysorbate 80, polyoxyl 40 hydrogenated castor oil, polyoxyl 35 castor oil, and mixtures thereof.

21. The pharmaceutical composition of claim 14, wherein said solubilizer comprises polysorbate 80.

22. The pharmaceutical composition of claim 14, wherein said diluent is selected from the group consisting of PEG 400, propylene glycol, propylene carbonate, triacetin, and mixtures thereof.

23. The pharmaceutical composition of claim 14, wherein said diluent comprises PEG 400.

24. The pharmaceutical composition of claim 14, wherein said stabilizer is a polyvinylpyrrolidone.

25. The pharmaceutical composition of claim 14, wherein said stabilizer is selected from polyvinylpyrrolidone 12, polyvinylpyrrolidone 17 and mixtures thereof.

26. The pharmaceutical composition of claim 14, wherein said carrier or excipient system comprises:
i) a viscosity builder selected from the group consisting of PEG 1000, PEG 1500, Gelucire 44/14, Gelucire 50/13, and mixtures thereof;
ii) a solubilizer selected from the group consisting of polysorbate 80, polyoxyl 40 hydrogenated castor oil, polyoxyl 35 castor oil, and mixtures thereof;
iii) a diluent selected from the group consisting of PEG 400, propylene glycol, propylene carbonate, Triacetin, and mixtures thereof; and
iv) a stabilizer comprising polyvinylpyrrolidone.

27. The pharmaceutical composition of claim 14, wherein said carrier or excipient system comprises:
i) PEG 1000 in an amount of from about 15% to about 25% by weight of the composition;
ii) polysorbate 80 in an amount of from about 5% to about 15% by weight of the composition;
iii) PEG 400 in an amount of from about 10% to about 50% by weight of the composition; and
iv) PVP K-17 in an amount of from about 1% to about 10% by weight of the composition.

28. A dosage form comprising a pharmaceutical composition of claim 14, wherein the composition contains from about 1 mg to about 125 mg of active pharmacological agent.

29. A dosage form comprising a pharmaceutical composition of claim 14, wherein the composition contains from about 3 mg to about 7 mg of active pharmacological agent.

30. A dosage form comprising a pharmaceutical composition of claim 14, wherein the composition contains from about 8 mg to about 12 mg of active pharmacological agent.

31. A dosage form comprising a pharmaceutical composition of claim 14, wherein the composition contains from about 13 mg to about 19 mg of active pharmacological agent.

32. A dosage form comprising a pharmaceutical composition of claim 14, wherein the composition contains from about 20 mg to about 30 mg of active pharmacological agent.

33. A dosage form comprising a pharmaceutical composition of claim 14, wherein the composition contains from about 31 mg to about 60 mg of active pharmacological agent.

34. A dosage form comprising a pharmaceutical composition of claim 14, wherein the composition contains from about 61 mg to about 80 mg of active pharmacological agent.

35. A dosage form comprising a pharmaceutical composition of claim 14, wherein the composition contains from about 81 mg to about 110 mg of active pharmacological agent.

36. A pharmaceutical composition comprising:
a) an active pharmacological agent comprising 4-(3-{5-chloro-1-(diphenylmethyl)-2-[2-({[2(trifluoromethyl)benzyl]sulfonyl}amino)ethyl]-1H-indol-3-yl}propyl)benzoic acid or a pharmaceutically acceptable salt thereof, in an amount of about 20% by weight of the composition; and
b) a carrier or excipient system comprising:
i) PEG 1000 in an amount of about 20% by weight of the composition;
ii) polysorbate 80 in an amount of about 10% by weight of the composition;
iii) PEG 400 in an amount of about 40% by weight of the composition; and
iv) PVP K-17 in an amount of about 10% by weight of the composition.

37. A dosage form comprising a pharmaceutical composition of claim 36, wherein said composition comprises about 100 mg of said active pharmacological agent.

38. A process for preparing a pharmaceutical composition comprising:
a) a pharmaceutically effective amount of an active pharmacological agent having the Formula II: 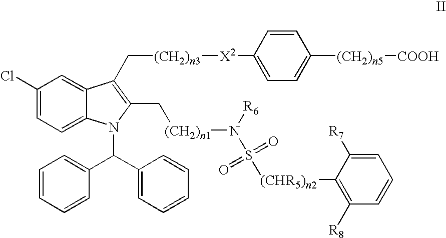
or a pharmaceutically acceptable salt thereof, wherein:
n₁ is 1 or 2;
n₂ is 1 or 2;
n₃ is 1 or 2;
n₅ is 0, 1 or 2;
X² is O, —CH₂— or SO₂;
each R₅ is independently H or C₁₋₃ alkyl;
R₆ is H or C₁₋₆ alkyl;
R₇ is selected from the group consisting of —OH, benzyloxy, —CH₃, —CF₃, —OCF₃, C₁₋₃ alkoxy, halogen, —CHO, —CO(C₁₋₃ alkyl), —CO(OC₁₋₃ alkyl), quinoline-5-yl, 3,5-dimethylisoxazol-4-yl, thiophene-3-yl, pyridin-4-yl, pyridine-3-yl, —CH₂-Q, and phenyl optionally substituted by from one to three independently selected R₃₀ groups;
R₈ is selected from the group consisting of H, —OH, —NO₂, —CF₃, —OCF₃, C₁₋₃ alkoxy, halogen, —CO(C₁₋₃ alkyl), —CO(OC₁₋₃ alkyl), quinoline-5-yl, 3,5-dimethylisoxazol-4-yl, thiophene-3-yl, —CH₂-Q, and phenyl substituted by from one to three independently selected R₃₀ groups;
Q is OH, dialkylamino, 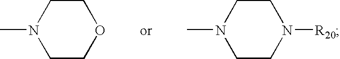
R₂₀ is selected from the group consisting of H, C₁₋₃ alkyl, and —CO(C₁₋₃ alkyl); and
R₃₀ is selected from the group consisting of dialkylamino, —CN and —OCF₃; provided that:
i) when each R₅ is H, R₆ is H, n₅ is 0, and R₈ is H, then R₇ cannot be chlorine;
ii) when each R₅ is H, R₆ is H, n₅ is 0, X² is O or —CH₂—, and R₈ is H, then R₇ cannot be CH₃;
iii) when each R₅ is H, and R₆ is H, then R₇ and R₈ cannot both be fluorine;
iv) when each R₅ is H, R₆ is H, and X² is O, then R₇ and R₈ cannot both be chlorine;
v) when each R₅ is H, R₆ is H, X² is O, and R₈ is NO₂, then R₇ cannot be fluorine; and
vi) when each R₅ is H, R₆ is H, X² is SO₂, and R₈ is H, then R₇ cannot be fluorine or chlorine; and
b) a carrier or excipient system comprising:
i) a viscosity builder comprising about 15% to about 25% by weight of the composition;
ii) a solubilizer comprising about 5% to about 15% by weight of the composition; and
iii) a diluent comprising about 10% to about 50% by weight of the composition; and
iv) a stabilizer comprising about 1% to about 10% by weight of the composition;
said process comprising
(1) mixing the viscosity builder, the solubilizer, and the diluent to produce a first homogenous solution;
(2) slowly adding the dispersing agent until dissolved to form a second homogenous solution;
(3) slowly adding the pharmacologically active agent to the second homogenous solution; and
(4) mixing with sufficient heating until the pharmacologically active agent is dissolved to produce a third homogenous solution.

39. The process of claim 38, wherein step (1) further comprises heating the viscosity builder, solubilizer, and diluent to a temperature sufficient to form the first homogenous solution.

40. The process of claim 39, wherein said mixing of the viscosity builder, solubilizer, and diluent is performed at a temperature of from about 90° C. to about 100° C.

41. The process of claim 39, further comprising the step of cooling the second homogenous solution from step (2) prior to slowly adding the pharmacologically active agent to the second homogenous solution in step (3).

42. The process of claim 41, wherein said second homogenous solution is cooled to about 80° C. to about 90° C.

43. The process of claim 38, wherein said mixing of said pharmacologically active agent in step (4) is performed at a temperature of from about 80° C. to about 90° C.

44. The process of claim 38, further comprising encapsulating at least a portion of said third homogenous solution into one or more unit dosage capsule forms.

45. The process of claim 44, wherein prior to encapsulation, said third homogenous solution is cooled.

46. The process of claim 45, wherein said third homogenous solution is cooled to about 40° C.

47. The process of claim 38, wherein said pharmaceutically effective amount of said active pharmacological agent is about 0.1% to about 25% by weight of the composition.

48. The process of claim 38, wherein said viscosity builder is selected from the group consisting of PEG 1000, PEG 1500, Gelucire 44/14, Gelucire 50/13, and mixtures thereof.

49. The process of claim 38, wherein said viscosity builder comprises PEG 1000.

50. The process of claim 38, wherein said solubilizer is selected from the group consisting of polysorbate 80, polyoxyl 40 hydrogenated castor oil, polyoxyl 35 castor oil, and mixtures thereof.

51. The process of claim 38, wherein said solubilizer comprises polysorbate 80.

52. The process of claim 38, wherein said diluent is selected from the group consisting of PEG 400, propylene glycol, propylene carbonate. Triacetin, and mixtures thereof.

53. The process of claim 38, wherein said diluent comprises PEG 400.

54. The process of claim 38, wherein said stabilizer comprises a polyvinylpyrrolidone.

55. The process of claim 38, wherein said stabilizer is selected from polyvinylpyrrolidone 12, polyvinylpyrrolidone 17 and mixtures thereof.

56. The process of claim 38, wherein said carrier or excipient system comprises:
i) a viscosity builder selected from the group consisting of PEG 1000, PEG 1500, Gelucire 44/14, Gelucire 50/13, and mixtures thereof;
ii) a solubilizer selected from the group consisting of polysorbate 80, polyoxyl 40 hydrogenated castor oil, polyoxyl 35 castor oil, and mixtures thereof;
iii) a diluent selected from the group consisting of PEG 400, propylene glycol, propylene carbonate, triacetin, and mixtures thereof; and
v) a stabilizer comprising a polyvinylpyrrolidone.

57. The process of claim 38, wherein said pharmaceutical composition comprising said carrier or excipient system comprises:
i) PEG 1000 in an amount of from about 15% to about 25% by weight of the composition;
ii) polysorbate 80 in an amount of from about 5% to about 15% by weight of the composition;
iii) PEG 400 in an amount of from about 10% to about 50% by weight of the composition; and
iv) PVP K-17 in an amount of from about 1% to about 10% by weight of the composition.

58. The process of claim 38, wherein the active pharmacological agent of Formula II has the Formula III: 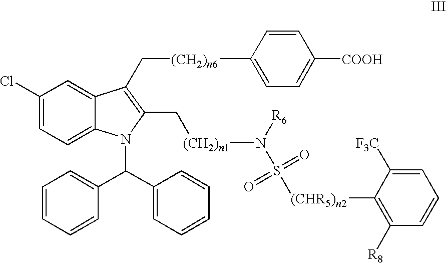
or a pharmaceutically acceptable salt thereof, wherein:
n₁ is 1 or 2;
n₂ is 1 or 2;
n₆ is 1 or 2;
R₅ is H or CH₃;
R₆ is H or C₁₋₆ alkyl; and
R₈ is selected from the group consisting of H, —OH, —NO₂, —CF₃, —OCF₃, —OCH₃, halogen, —COCH₃, —COOCH₃, dimethylamino, diethylamino and —CN.

59. The process of claim 38, wherein the active pharmacological agent comprises 4-(3-{5-chloro-1-(diphenylmethyl)-2-[2-({[2-(trifluoromethyl)benzyl]sulfonyl}amino)ethyl]-1H-indol-3-yl}propyl)benzoic acid or a pharmaceutically acceptable salt thereof.

60. A process for preparing a pharmaceutical composition comprising:
a) a pharmacologically active agent comprising 4-(3-{5-chloro-1-(diphenylmethyl)-2-[2-({[2-(trifluoromethyl)benzyl]sulfonyl}amino)ethyl]-1H-indol-3-yl}propyl)benzoic acid or a pharmaceutically acceptable salt thereof, in an amount of about 20% by weight of the composition; and
b) a carrier or excipient system comprising:
i) PEG 1000 in an amount of about 20% by weight of the composition;
ii) polysorbate 80 in an amount of about 10% by weight of the composition;
iii) PEG 400 in an amount of about 40% by weight of the composition; and
iv) PVP K-17 in an amount of about 10% by weight of the composition;
said process comprising
(1) mixing the PEG 1000, polysorbate 80, and PEG 400 to produce a first homogenous solution;
(2) slowly adding PVP K-17 until dissolved to form a second homogenous solution;
(3) slowly adding the pharmacologically active agent to the second homogenous solution;
(4) mixing with sufficient heating until the pharmacologically active agent is dissolved to produce a third homogenous solution.

61. The process of claim 60, wherein step (1) further comprises heating the PEG 1000, polysorbate 80, and PEG 400 to a temperature sufficient to form the first homogenous solution.

62. The process of claim 61, wherein said mixing of the PEG 1000, polysorbate 80, and PEG 400 is performed at a temperature of from about 90° C. to about 100° C.

63. The process of claim 61, further comprising the step of cooling the second homogenous solution from step (2) prior to slowly adding the pharmacologically active agent to the second homogenous solution in step (3).

64. The process of claim 63, wherein said second homogenous solution is cooled to about 80° C. to about 90° C.

65. The process of claim 60, wherein said mixing of said pharmacologically active agent in step (4) is performed at a temperature of from about 80° C. to about 90° C.

66. The process of claim 64, further comprising encapsulating at least a portion of the third homogenous solution into one or more unit dosage capsule forms.

67. A process of claim 66, wherein prior to encapsulation, the third homogenous solution is cooled.

68. A process of claim 67, wherein said third homogenous solution is cooled to about 40° C.

69. A process of claim 60, wherein said encapsulated third homogenous solution comprises about 100 mg of said active pharmacological agent.

70. A product made by the process of claim 38.
